(19) United States
(12) Patent Application Publication
(10) Pub. No.: US 2026/0108382 A1
(43) Pub. Date: Apr. 23, 2026

(54) MANDIBULAR ADVANCEMENT DEVICE WITH ENHANCED STABILITY

(71) Applicant: DCSTAR INC, New York, NY (US)

(72) Inventor: David LUO, New York, NY (US)

(21) Appl. No.: 18/924,719

(22) Filed: Oct. 23, 2024

(51) Int. Cl. A61F 5/56; A61C 5/00

(52) U.S. Cl. CPC A61F 5/566; A61C 5/007

(57) ABSTRACT

A mandibular advancement device with enhanced stability, including an upper tray assembly formed by an upper moldable component and an upper bracket, and a lower tray assembly formed by a lower moldable component and a lower bracket. The upper bracket and the lower bracket each extend a front wall from their front side edges near the labial side, while at least the lower bracket extends a rear wall along the median sagittal plane from the rear side edge near the lingual side. The stability is achieved by inclining the front wall of at least the upper bracket towards the lingual side. The rear wall of the lower bracket works in conjunction with the front wall to clamp the lower dental arch. Additionally, the stability feature includes a molar clamping section, further enhancing the overall stability of the mandibular advancement device.

### TECHNICAL FIELD

The disclosure relates to a mandibular advancement device with enhanced stability. The design of this mandibular advancement device allows it to remain securely in the patient's oral cavity during sleep, thereby increasing patient comfort and treatment adherence.

### BACKGROUND

Obstructive Sleep Apnea (OSA) is a common sleep disorder in modern society, characterized by repeated episodes of breathing cessation and hypoventilation during sleep. Sleep disorders affect hundreds of millions of people worldwide, with approximately 50 to 70 million individuals in the United States suffering from chronic sleep or wakefulness disorders. This condition is extremely detrimental to mental and physical health, potentially leading to a range of complications such as depression, mania, bipolar disorder, and schizophrenia.

Snoring is often a symptomatic manifestation of OSA and may not be merely a harmless habit. If snoring is accompanied by symptoms such as breathing pauses, daytime sleepiness, and frequent nighttime awakenings, it is advisable to seek medical evaluation and diagnosis promptly. Appropriate treatment measures can then be taken to improve sleep quality and overall health. Research shows that the prevalence of habitual snoring in European populations ranges from 15.6% to 19%, with occasional snoring at 26% to 30%. In Japan, the prevalence of snoring ranges from 12.8% to 16.0%. Surveys in China indicate a snoring prevalence of approximately 13%, which increases with age. Among individuals aged 60 to 69, the prevalence is 39% for men and 17% for women. When using AHI≥5 as the diagnostic criterion for OSAS, the sensitivity and specificity of OSAS prevalence rates are 70.8% and 47.7%, respectively. In the United States, the prevalence of OSAS among men over the age of 40 is 1.24%, while in European countries it ranges from 1% to 2.7%, and in Japan, from 1.3% to 4.2%. Overall, 14% of men and 5% of women suffer from OSAS, with an increasing trend observed. The prevention and treatment of snoring and/or obstructive sleep apnea are becoming increasingly urgent issues.

Mandibular advancement devices are therapeutic appliances specifically designed to address symptoms such as OSA that occur during sleep. The treatment principle of a mandibular advancement device involves the device being divided into upper and lower parts, which work together to reposition the patient's mandible forward through offset fixation of these two parts. Before treatment, the patient needs to select an appropriate mandibular adjustment position for themselves. During sleep, the device is placed in the mouth to maintain this selected position, thereby increasing the cross-sectional area of the upper airway. This helps keep the airway open during sleep, preventing airway collapse and obstruction, and thereby improving the patient's breathing condition. Currently, mandibular advancement devices are available in two types: custom-made and non-custom-made. Each has its own advantages and characteristics. Custom-made mandibular advancement devices are tailored to fit the patient's oral structure based on their dental model, providing a perfect fit and thus greater comfort. However, the customization process usually requires the full involvement of a dental professional and incurs higher costs due to the personalized manufacturing. Therefore, for patients with budget constraints or those who need an immediate solution, non-custom-made mandibular advancement devices are a more suitable option.

When choosing between custom-made and non-custom-made mandibular advancement devices, several factors should be considered, including the patient's budget, the severity of OSA, the urgency of treatment, and the patient's treatment preferences. These considerations will help determine which type of device is most suitable for the patient.

### SUMMARY

This disclosure aims to provide a mandibular advancement device that is convenient for patients to use and comfortable to wear.

In one embodiment, a mandibular advancement device with enhanced stability is provided. The mandibular advancement device includes an upper tray assembly and a lower tray assembly. The upper tray assembly is configured to be positioned adjacent to an upper dental arch of a patient when placed inside the patient's mouth, while the lower tray assembly is configured to be positioned adjacent to a lower dental arch of the patient when placed inside the patient's mouth. The upper tray assembly includes an upper moldable component and an upper bracket, with different required temperatures for heating the upper moldable component and the upper bracket to deform. The lower tray assembly includes a lower moldable component and a lower bracket, with different required temperatures for heating the lower moldable component and the lower bracket to deform. The upper bracket and the lower bracket each have a front side edge configured to be near a labial side and a rear side edge configured to be near a lingual side, with at least the upper bracket having a protruding front wall on the front side edge, and at least the lower bracket having a protruding rear wall on the rear side edge. The rear wall of at least the lower bracket includes a baffle section at the median sagittal plane, and at least a portion of the front wall of at least the upper bracket is inclined towards the lingual side.

In one embodiment, the upper moldable component and the lower moldable component are configured to soften when heated in hot water, allowing them to be adjusted and molded to match the shapes of the upper and lower dental arches of the patient, respectively, when the patient bites.

In one embodiment, the upper moldable component and the lower moldable component include a flexible thermoplastic material.

In one embodiment, the hardness of the material of the upper bracket is at least partially greater than that of the upper moldable component, and the hardness of the material of the lower bracket is at least partially greater than that of the lower moldable component.

In one embodiment, the upper bracket and the lower bracket each have a bottom wall that is configured to be opposite an occlusal surface of teeth when the patient bites, and the bottom wall of the upper bracket and the bottom wall of the lower bracket are at least partially in contact when placed inside the mouth of the patient.

In one embodiment, the length of the baffle section provided at the rear side edge is greater than 10% of the total length of the rear side edge of the lower bracket.

In another embodiment, a mandibular advancement device with enhanced stability is provided. A mandibular advancement device with enhanced stability includes an upper tray assembly and a lower tray assembly. The upper tray assembly has an arcuate outer shape configured to conform to an upper dental arch curve of a patient. The lower tray assembly has an arcuate outer shape configured to conform to a lower dental arch curve of the patient. The upper tray assembly includes an upper moldable component and an upper bracket. Required temperatures at which the upper moldable component and the upper bracket are heated to deform differ. The lower tray assembly includes a lower moldable component and a lower bracket. Required temperatures at which the lower moldable component and the lower bracket are heated to deform differ. The upper bracket and the lower bracket each have a front side edge configured to be near a labial side and a rear side edge configured to be near a lingual side. The upper bracket and the lower bracket each have a protruding front wall on the front side edges and a protruding rear wall on the rear side edges. The front wall and the rear wall of at least the upper bracket include a clamping section configured to be positioned near a molar area when placed inside the mouth of the patient. The rear wall of at least the lower bracket further includes a baffle section at a median sagittal plane.

In one embodiment, the arcuate outer shape of the upper tray assembly and the lower tray assembly includes at least one of an oval arch, a conical arch, or a square arch.

In one embodiment, the baffle section is divided into multiple spaced-apart sections. In one embodiment, at least the lower moldable component has a shape configured to engage and match with the baffle section.

In one embodiment, the upper moldable component and the lower moldable component are made of a moldable material, and a temperature required to heat them to deform is less than 100 degrees Celsius.

In one embodiment, the upper bracket and the lower bracket are made of a material with high thermal stability, and a temperature required to heat them to deform is greater than 100 degrees Celsius.

In yet another embodiment, a mandibular advancement device with enhanced stability is provided. A mandibular advancement device with enhanced stability includes an upper tray assembly and a lower tray assembly. The upper tray assembly is configured to be positioned adjacent to an upper dental arch of a patient when placed inside the mouth of the patient. The lower tray assembly is configured to be positioned adjacent to a lower dental arch of the patient when placed inside the mouth of the patient. The upper tray assembly includes an upper moldable component and an upper bracket, with different required temperatures for heating the upper moldable component and the upper bracket to deform. The lower tray assembly includes a lower moldable component and a lower bracket, with different required temperatures for heating the lower moldable component and the lower bracket to deform. The upper bracket and the lower bracket each have a front side edge configured to be near a labial side and a rear side edge configured to be near a lingual side. The upper bracket and the lower bracket each have a protruding front wall on the front side edges and a protruding rear wall on the rear side edges. The front wall and the rear wall of at least the upper bracket include a clamping section configured to be positioned near a molar area when placed inside the mouth of the patient. At least one distance between the front wall and the rear wall at the clamping section is greater than a corresponding shortest distance between the front wall and the rear wall at other areas on the upper bracket, and a ratio of the two distances is at least 1.12:1. At least a portion of the front wall of at least the upper bracket is inclined towards the lingual side.

In one embodiment, the upper bracket and the lower bracket are respectively configured to engage and support the upper moldable component and the lower moldable component.

In one embodiment, a connection module is provided between the upper bracket and the lower bracket.

In one embodiment, the upper bracket and the lower bracket each have a bottom wall configured to be opposite an occlusal surface of teeth when the patient bites, and the connection module includes a position adjustment snap-fit provided on the bottom wall.

In one embodiment, the connection module includes a protrusion provided on the front wall and/or rear wall of the upper bracket and/or the lower bracket.

In a further embodiment, a mandibular advancement device with enhanced stability is provided. A mandibular advancement device with enhanced stability includes an upper tray assembly and a lower tray assembly. The upper tray assembly has an arcuate outer shape configured to conform to an upper dental arch curve of a patient. The lower tray assembly has an arcuate outer shape configured to conform to a lower dental arch curve of the patient. The upper tray assembly includes an upper moldable component and an upper bracket, with different required temperatures for heating the upper moldable component and the upper bracket to deform. The lower tray assembly includes a lower moldable component and a lower bracket, with different required temperatures for heating the lower moldable component and the lower bracket to deform. The upper bracket and the lower bracket each have a front side edge configured to be near a labial side and a rear side edge configured to be near a lingual side, with at least the upper bracket having a protruding front wall on the front side edge, and at least the lower bracket having a protruding rear wall on the rear side edge. The rear wall of at least the lower bracket includes a baffle section at a median sagittal plane. The front wall of at least the upper bracket has an outer wall configured to face towards the labial side and an inner wall opposite to the outer wall, and an angle between the inner wall of the front wall of at least the upper bracket at a median sagittal plane and a horizontal plane is less than 90°. A height of the front wall of at least the upper bracket is less than 19.5 mm.

In one embodiment, the upper tray assembly and the lower tray assembly are in a form that is higher in the middle and lower on both sides.

In one embodiment, in the lower tray assembly, a maximum height of the baffle section is less than or equal to a maximum height of the lower moldable component.

In one embodiment, the upper tray assembly and the lower tray assembly are made of at least two different materials.

In one embodiment, a projected area of the upper moldable component on a horizontal plane is greater than a projected area of the upper bracket on the same plane, and a projected area of the lower moldable component on a horizontal plane is greater than a projected area of the lower bracket on the same plane.

The implementation of a mandibular advancement device provided by this disclosure has at least the following beneficial effects:

1. Through comprehensive research, design, and experimentation, this disclosure introduces a novel mandibular advancement device that aligns with the oral structure of the human body while further enhancing its effectiveness and stability. Initial analysis of the current market revealed several areas where existing mandibular advancement devices need improvement, particularly with non-custom-made devices, which frequently exhibit the problem of falling out when the mouth opens. Feedback collected from various e-commerce platforms showed that non-custom-made mandibular advancement devices generally received poor reviews. Following a detailed assessment of patient needs and an extensive analysis of oral structure data, several key factors contributing to these issues were identified across different patient groups'oral structures and usage habits: a. The upper tray assembly tends to fall downward under the influence of gravity; b. The outer surface of the patient's central incisors does not fit well with the moldable component; c. The upper and lower tray assemblies do not adequately conform to the patient's molars, posing a risk of dislodgement; d. when the patient bites, the lower moldable component is susceptible to forces exerted by the mandible returning to its original position, causing it to separate from the lower bracket and rendering the device ineffective. Each of these issues was analyzed to identify the underlying causes, and corresponding solutions were developed. Extensive testing was then conducted, leading to the development of a more effective mandibular advancement device grounded in both theory and practice. (1) The occlusion between the upper and lower dental arches is essentially the rotation and movement of the mandible in the glenoid fossa around the condyle, causing the mandible to move towards the maxilla. Therefore, during the shaping process of the upper moldable component, the lower dental arch pushes both the upper and lower tray assemblies upward until the upper moldable component compresses against the upper dental arch. However, because the outer surface of the central incisors is slanted rather than vertical, and the upper moldable component is compressed upward vertically, the surface it presses against becomes a vertical inner surface. This difference in angles creates a gap, preventing the upper moldable component from properly fitting the upper dental arch, which leads to the upper tray assembly falling out. To address this, the design includes a front wall with an inward inclination, which pushes the overflowing material towards the outer surface of the central incisors during the upward compression of the upper tray assembly. This ensures that the upper moldable component conforms perfectly to the upper dental arch during the shaping process. The inwardly inclined front wall also works in conjunction with the rear wall to securely hold the central incisors. The combination of the inclined front and rear walls provides a more secure clamp on the central incisors, ensuring the overall stability of the mandibular advancement device. (2) Molars are relatively short, and their contact surface with the upper moldable component and the lower moldable component is insufficient, which, combined with the weight of the upper tray assembly, often causes the mandibular advancement device to fall out at the molar region. To improve the stability of the upper and lower tray assemblies when placed inside the patient's mouth, at least the upper bracket includes a front wall and a rear wall positioned to contact the patient's teeth. Nearer the molar areas, the front wall and the rear wall form a clamping section that, during the molding process of the upper moldable component and the lower moldable component when the patient bites down, helps direct the overflowing material upwards, increasing the contact surface area between the moldable components and the upper and lower dental arches. Additionally, the clamping section helps secure the molars, making the connection between the upper and lower tray assemblies more stable. (3) The working principle of the mandibular advancement device involves fixing the relative positions of the upper and lower tray assemblies to control the relative positioning of the upper and lower dental arches for corrective purposes. Although the lower tray assembly moves the lower dental arch forward, this movement opposes the natural position of the mandible, causing a backward force to act on the lower tray assembly during use. This force typically acts on the lower moldable component. If the connection between the lower moldable component and the lower bracket is not secure, the lower moldable component may shift relative to the lower bracket, leading to device failure. To address this, a rear wall is set at the median sagittal plane of at least the lower bracket, further forming what is referred to as a baffle section. The baffle section provides resistance against the dislodging force acting on the lower moldable component, maintaining its fixed position relative to the lower bracket and ensuring the device remains effective. (4) After incorporating the above structures to enhance stability, repeated experimental testing and multiple iterative optimizations were carried out to mitigate the influence of gravity on the upper tray assembly and enhance wearing comfort. This refined structure allows the device to maximize its effectiveness while ensuring patient comfort during use.

2. Dentistry is a precise and data-driven field, where even small variations in data-such as angle, weight, height, width, and material flexibility-can affect patient comfort and device effectiveness. This disclosure carefully fine-tunes these parameters through repeated testing to achieve optimal performance and better comfort for patients. By refining the design structure and conducting detailed data analysis and calibration, the disclosure results in a more comfortable mandibular advancement device, which improves patient treatment adherence. (1) the upper bracket and the lower bracket are designed to be wider at the clamping section compared to other areas. This improvement is based on the fact that molars are typically wider than other teeth in the oral structure, yet existing mandibular advancement devices do not adjust the width of the clamping section accordingly. The widened clamping section allows the upper moldable component and the lower moldable component to better conform to the patient's upper and lower dental arches after molding, ensuring a secure but not overly tight clamp on the molars, enhancing comfort. (2) The baffle section of at least the lower bracket is designed with an angle that aligns with the natural shape of the gums and teeth. Since the gums and lower teeth generally do not have vertical surfaces but have sloped ones, the angled baffle section matches the slope of the gums and teeth. This design allows the mandibular advancement device to fit more snugly within the patient's mouth, enhancing comfort during use. Comfort is a critical factor for patients-if the device causes discomfort or issues such as component dislodgement during use, patients may be less willing to wear it for extended periods, which could negatively impact treatment outcomes. By improving comfort, this disclosure increases patient adherence and contributes to more stable and effective treatment. (3) Additionally, this disclosure incorporates specific parameter adjustments to improve the effectiveness of the mandibular advancement device during use. For example, the height of the front wall is limited to ensure that it can securely hold and press against the upper and/or lower dental arches without scraping the gums. The angle of the front wall is also specified to allow the front wall of at least the upper bracket to tilt the upper moldable component towards the lingual side when the patient bites, without clamping the upper dental arch too tightly and causing discomfort. The length of the baffle section is controlled to ensure it provides effective support and stabilization.

3. The addition of the baffle section extends the lifespan of the mandibular advancement device, providing cost-saving benefits for the patient. Devices without a baffle section often face issues like the lower moldable component becoming dislodged, which can easily lead to damage since the upper moldable component and the lower moldable component are made from flexible materials. This increases the frequency and cost of replacing these parts for the patient. Damage not only shortens the device's lifespan but also undermines its stability, weakening its therapeutic effectiveness. In contrast, the mandibular advancement device in this disclosure features a baffle section that strengthens the connection between the lower moldable component and the lower bracket. This design effectively prevents the force from pulling the lower moldable component out of place, ensuring that the device remains securely in place during use and reducing the risk of damage. As a result, the device's stability and durability are improved while maintaining a reliable and effective treatment.

4. Typically, while custom-made mandibular advancement devices offer a higher degree of fit with the teeth and allow for breathing through the mouth, they require technologies like oral scanning for customization. This process incurs significant labor and time costs, and a set of custom-made mandibular advancement devices generally costs patients thousands of dollars. In comparison, the mandibular advancement device provided by this disclosure has several advantages over custom-made devices. a. The mandibular advancement device in this disclosure provides the same benefits as custom-made devices, such as allowing mouth-breathing and reducing the risk of dislodgement. Through features like an inclined front wall, the inclusion of a baffle section, and clamping sections, along with precise structural parameters, this design improves the treatment's effectiveness while ensuring stability throughout the patient's therapy. b. The mandibular advancement device of this disclosure is more cost-effective than custom-made devices, reducing the financial burden on patients. This allows patients with limited budgets to access a high-quality mandibular advancement device. While custom-made devices often require precise instruments and labor, driving up costs, the design and production of this disclosure enable patients to achieve the effects of a custom-made device at a lower cost, making it a more practical and economical choice for certain patient groups. c. The mandibular advancement device of this disclosure can be produced and used quickly, saving patients the wait time required for custom-made devices. This makes it a better option for patients seeking prompt treatment. d. In the market, only custom-made mandibular advancement devices typically offer the ability to open and close the mouth during use. However, the non-custom mandibular advancement device of this disclosure achieves the same functionality, meeting a wider range of patient needs. For example, patients who habitually sleep with their mouths open or those who are prone to snoring during sleep benefit from the device's ability to accommodate mouth opening, offering unique advantages. It not only provides greater comfort but also attracts a broader consumer base. e. The main body of custom-made mandibular advancement devices (i.e., the part in direct contact with the teeth) is typically made of rigid, one-piece material. In contrast, the mandibular advancement device of this disclosure uses flexible materials in the sections that contact the inside of the patient's mouth. This prevents irritation to the oral tissues, reducing the sensation of a foreign object while wearing the device. Additionally, in the event of accidental impacts, the flexible material acts as a buffer, protecting the teeth. This makes the device a safer option compared to traditional designs.

### BRIEF DESCRIPTION OF THE DRAWINGS

FIG. 1 is an overview diagram of the human respiratory system, including the nasal cavity, oral cavity, larynx, esophagus, trachea, and bronchi;

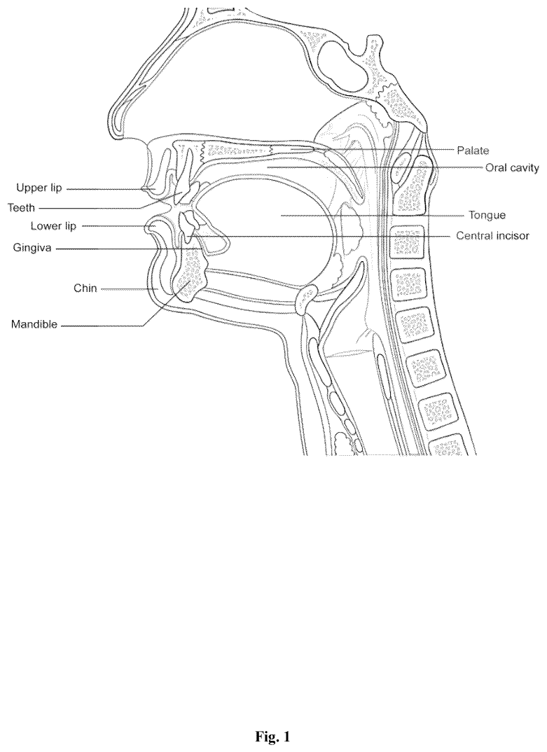

FIG. 2 is an anatomical overview diagram of the human oropharyngeal region, showing several identified surface anatomical features, including the oral cavity, pharynx, cervical vertebrae, salivary glands, and mandible;

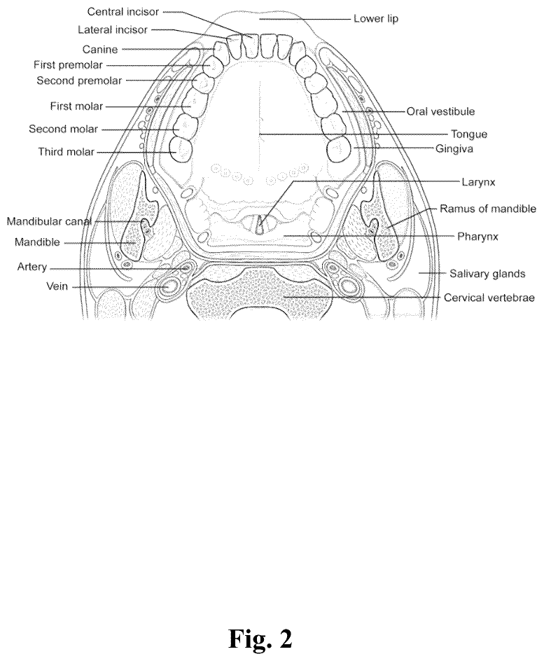

FIG. 3 is an overview diagram of the interior of the human oral cavity, showing several identified surface anatomical features, including teeth, palate, pharynx, tongue, and gingiva;

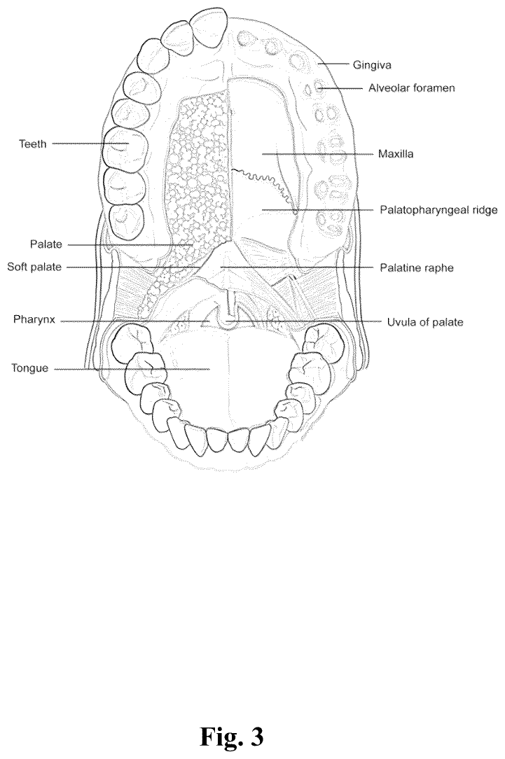

FIG. 4 is a side view of the human skull, including the mandible, teeth, vertebrae, larynx, maxilla, and condyle;

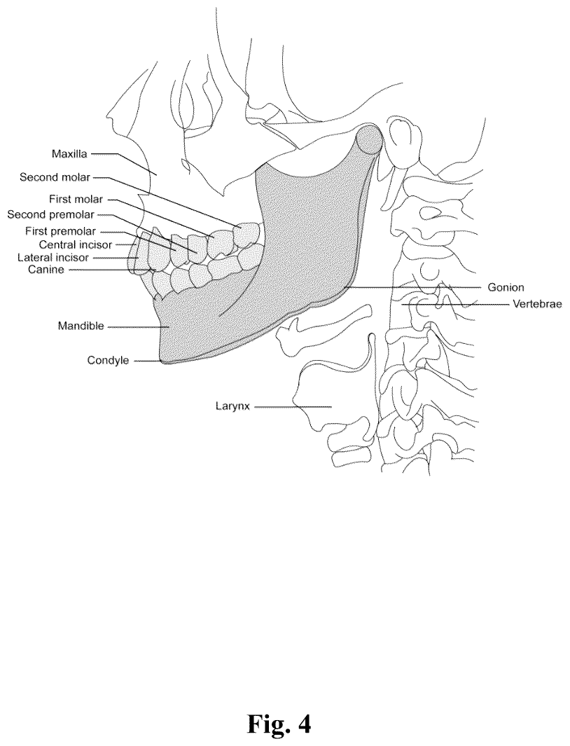

FIG. 5 is a side view of the human skull and oral muscles, including the buccinator muscle, orbicularis oris muscle, labial part of orbicularis oris muscle and marginal part of orbicularis oris muscle;

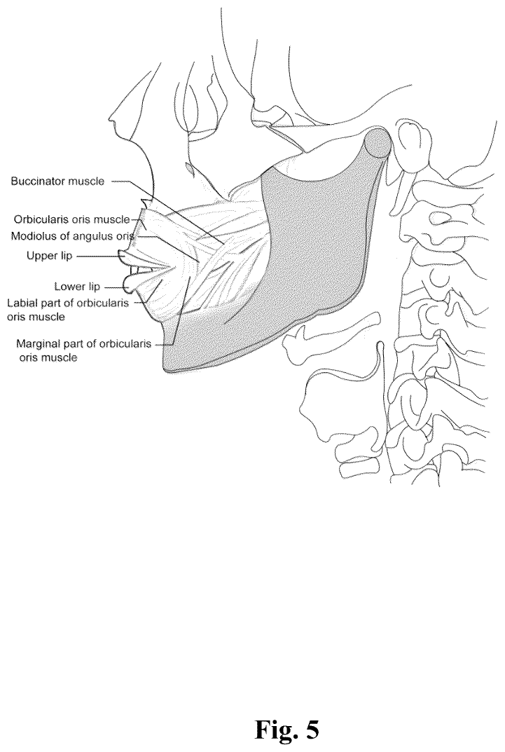

FIG. 6 is a side view of the mandibular structure within the human head, showing the approximate position of the upper dental arch and mandible;

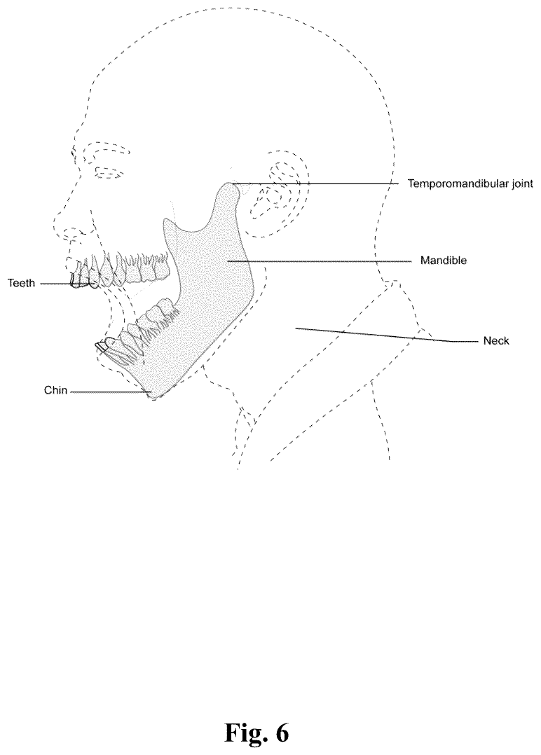

FIG. 7 is a schematic diagram of the specific structure of the mandible, including the teeth, chin, neck of mandible, condylar process, and mandibular condyle;

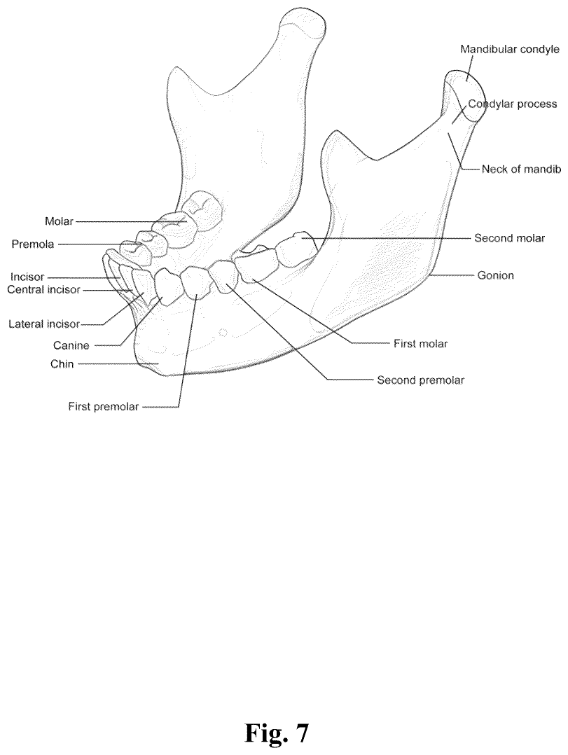

FIG. 8 is an anatomical overview diagram of the mandible, including the teeth, body of the mandible, superior mental spines, and mandibular torus;

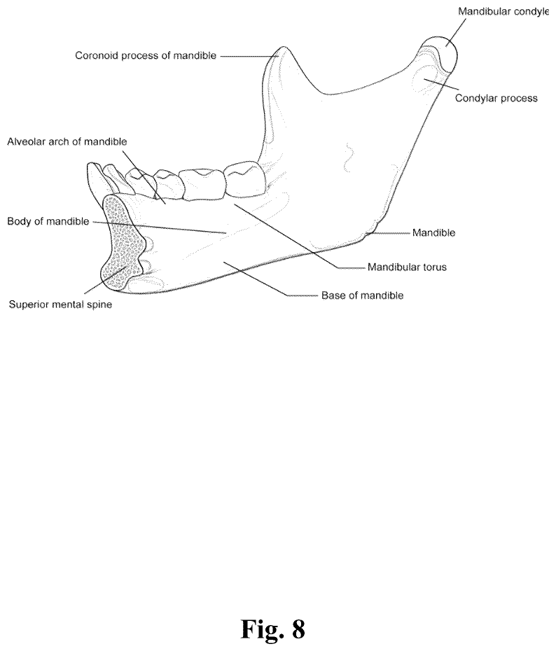

FIG. 9 is a schematic diagram of the movement of the mandible relative to the maxilla, showing the rotation and movement of the mandible with the condyle as the pivot point;

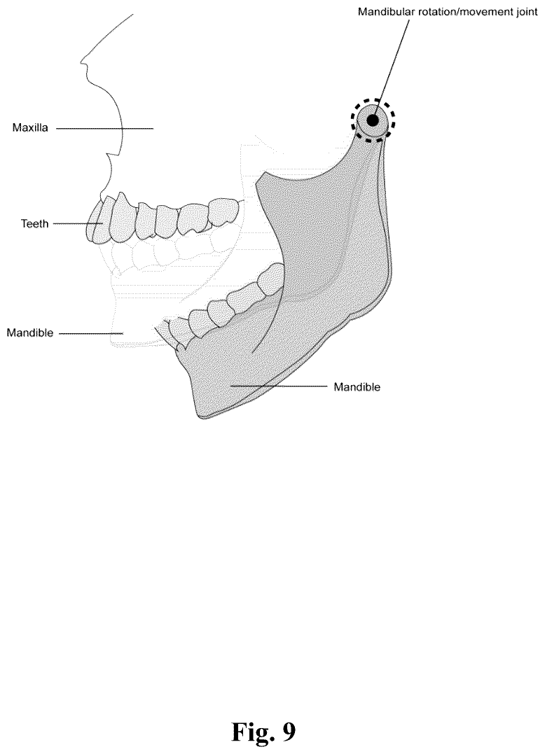

FIG. 10 is an overall schematic diagram of the mandibular advancement device in accordance with one embodiment;

FIG. 11 is an exploded structural diagram of the mandibular advancement device in accordance with one embodiment;

FIGS. 12A, 12B and 12C are a top view of the upper tray assembly and the shape of the upper dental arch in accordance with multiple embodiments;

FIG. 13 is a schematic diagram of the horizontal plane, sagittal plane, and coronal plane of the mandibular advancement device in multiple embodiments;

FIG. 14 is a schematic diagram showing the front side edge and the rear side edge of the mandibular advancement device in multiple embodiments;

FIG. 15 is a schematic diagram showing the front wall with a certain inclination angle of the mandibular advancement device in accordance with one embodiment;

FIG. 16 is a schematic diagram showing the rear wall with a certain inclination angle of the mandibular advancement device in accordance with one embodiment;

FIG. 17 is a schematic diagram showing the separation of the lower moldable component from the lower bracket in the mandibular advancement device in multiple embodiments;

FIG. 18 is a schematic diagram showing the molding process of the upper moldable component with the upper dental arch in an existing mandibular advancement device;

FIG. 19 is a schematic diagram showing the molding process of the upper moldable component with the upper dental arch in multiple embodiments;

FIG. 20 is a top view of the mandibular advancement device in multiple embodiments;

FIG. 21 is a top view of the upper moldable component and the upper bracket in multiple embodiments;

FIGS. 22A, 22B and 22C are schematic diagrams of various forms of the baffle section in accordance with one embodiment;

FIG. 23 is a schematic diagram of the upper bracket and the lower bracket in accordance with one embodiment;

FIG. 24 is a schematic diagram of the closed state of the mandibular advancement device with a connection module in the form of a protrusion in multiple embodiments;

FIG. 25 is a schematic diagram of the open state of the mandibular advancement device with a connection module in the form of a protrusion in multiple embodiments;

FIG. 26 is a schematic diagram of the upper bracket or lower bracket with a clamping section in multiple embodiments;

FIG. 27 is a schematic diagram of the height of the front wall at the median sagittal plane and the length of the clamping section in multiple embodiments;

FIG. 28 is a schematic diagram of the distance between the front wall and the rear wall at the median sagittal plane and the clamping section in multiple embodiments;

FIG. 29 is a top view of the upper bracket or lower bracket in multiple embodiments;

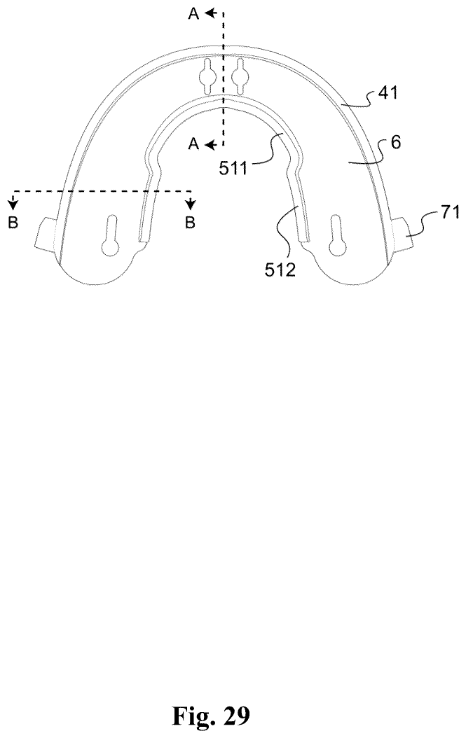

FIG. 30 is a schematic diagram showing the height and width differences in the clamping section at cross-sectional positions A-A and B-B in FIG. 29 in multiple embodiments;

FIGS. 31A, 31B and 31C are schematic diagrams of various forms of the clamping section in accordance with one embodiment;

FIG. 32 is a schematic diagram of the baffle section and clamping section being two separate parts in accordance with one embodiment;

FIG. 33 is a schematic diagram of the baffle section having multiple spaced-apart walls in accordance with one embodiment;

FIG. 34 is a schematic diagram of the connection module in the form of a snap-fit in accordance with one embodiment;

FIG. 35 is a schematic diagram of the connection of the snap-fit-type connection module in accordance with one embodiment.

### DETAILED DESCRIPTION

To elucidate the objectives, features, and advantages of this disclosure more clearly, a detailed description of specific embodiments of the disclosure is provided below in conjunction with the accompanying drawings. Many specific details are provided in this description to facilitate a comprehensive understanding of the disclosure. However, it is feasible to implement the disclosure in various ways other than those described here, and one skilled in the art can make similar modifications without departing from the essence of the disclosure. Therefore, the disclosure is not confined to the specific embodiments disclosed herein.

Compared to existing mandibular advancement devices on the market, this disclosure provides a detailed design and adjustment of the front and rear walls of the upper bracket and the lower bracket. The front wall of at least the upper bracket is inclined inward, and an additional rear wall is added at the median sagittal plane of at least the lower bracket. These improvements make the mandibular advancement device of this disclosure more stable. Additionally, the device includes a clamping section near the point of contact with the molars, and at least one protrusion is provided on the upper bracket and/or the lower bracket. The design of these structures improves the stability of the mandibular advancement device, improving both its effectiveness and comfort during use. This prevents the device from dislodging during sleep, thereby increasing patient compliance and enhancing the device's effectiveness, comfort, reliability, and durability. As a result, this mandibular advancement device not only improves performance metrics but also provides patients with a more stable, safe, and comfortable experience.

This disclosure offers a mandibular advancement device 1 that is easy for patients to use and and provides comfort during wear.

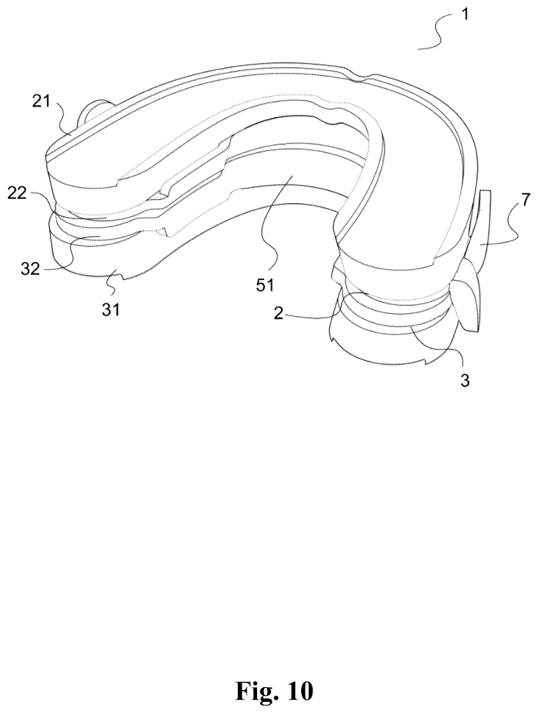

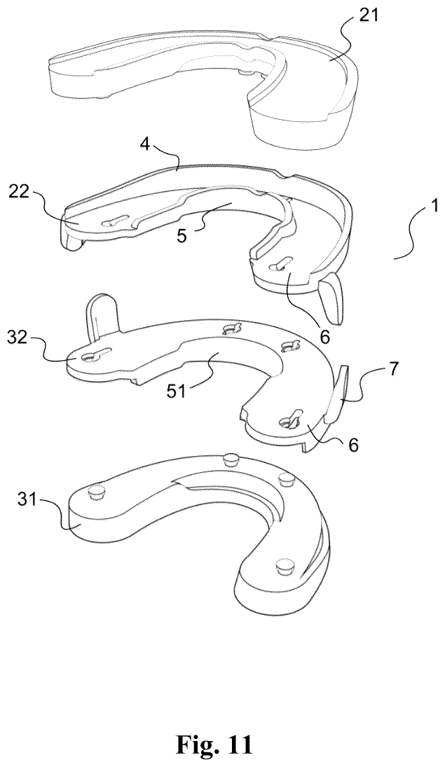

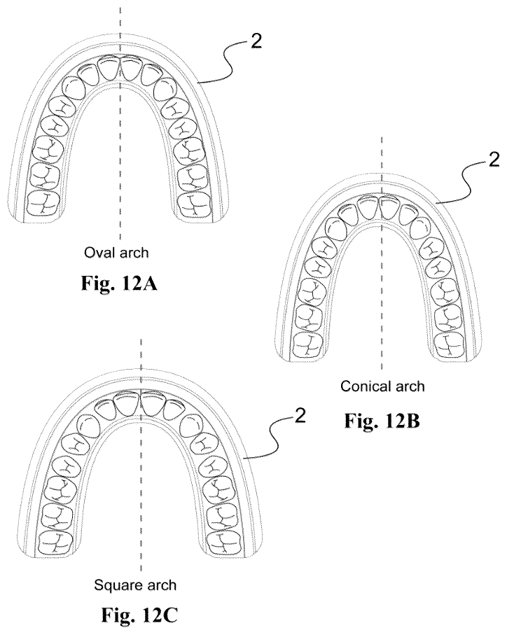

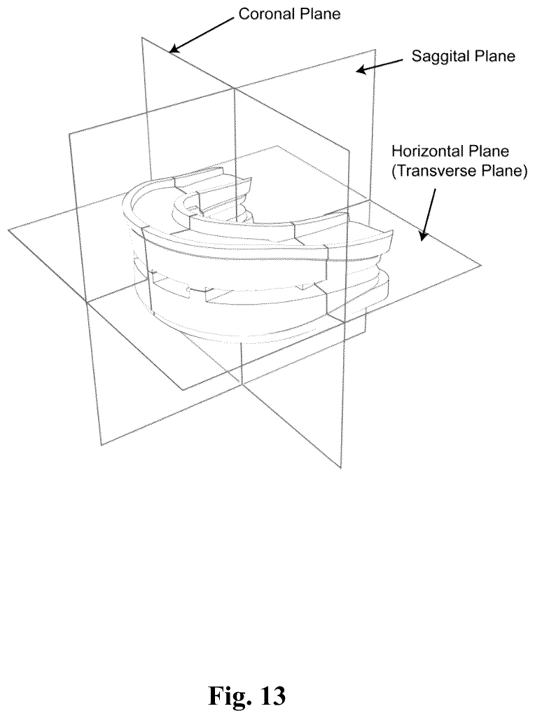

Specifically, as referenced in FIGS. 10-21, the mandibular advancement device 1 of the present disclosure includes an upper tray assembly 2 and a lower tray assembly 3. The upper tray assembly 2 has an arcuate outer shape configured to conform to the curve of the patient's upper dental arch and is positioned adjacent to the upper dental arch when placed in the patient's mouth. The lower tray assembly 3 has an arcuate outer shape configured to conform to the curve of the patient's lower dental arch and is positioned adjacent to the lower dental arch when placed in the patient's mouth. When the mandibular advancement device 1 is positioned in the patient's mouth, the upper tray assembly 2 and lower tray assembly 3 are configured to fit, at least partially, against the patient's upper and lower dental arches during use. The shape of the dental arch can generally be categorized into oval, conical, or square arches. Therefore, the arcuate shape of the upper tray assembly 2 and lower tray assembly 3 adopts at least one of an oval arch, a conical arch, or a square arch, as shown in FIGS. 12A, 12B and 12C.

Furthermore, the upper tray assembly 2 includes an upper moldable component 21 and an upper bracket 22, while the lower tray assembly 3 includes a lower moldable component 31 and a lower bracket 32. The upper moldable component 21 and the lower moldable component 31 are configured to at least partially contact the patient's upper and lower dental arches when the mandibular advancement device 1 is positioned in the patient's mouth. The upper moldable component 21 and the lower moldable component 31 have surfaces that contact the teeth, which may include at least one groove to accommodate any overflowing material during the patient's bite, allowing for a better fit with the upper and lower dental arches. In some cases, the surfaces of the upper moldable component 21 and/or the lower moldable component that contact the teeth may be flat or curved without grooves.

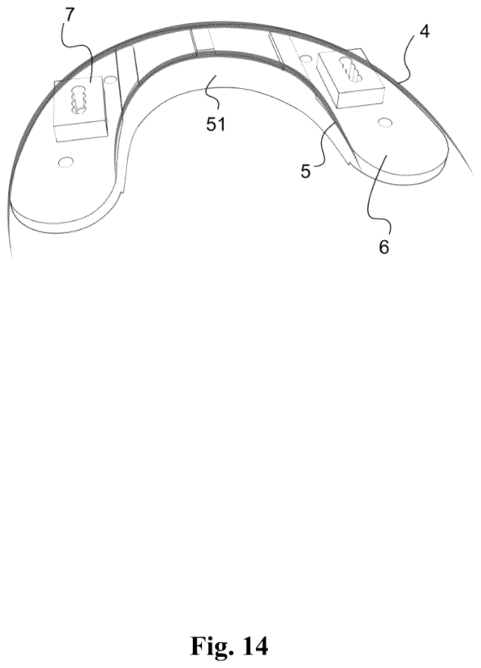

The upper bracket 22 and the lower bracket 32 are each configured to engage and support the upper moldable component 21 and the lower moldable component 31, respectively. Both the upper bracket 22 and the lower bracket 32 have a bottom wall 6 positioned opposite the occlusal surface of the teeth when the patient bites. When placed in the patient's mouth, the bottom wall 6 of the upper bracket 22 and the bottom wall 6 of the lower bracket 32 are at least partially in contact. The upper bracket 22 and the lower bracket 32 have a front side edge 4 near the labial side and a rear side edge 5 near the lingual side (as shown in FIG. 14). During treatment, the mandibular advancement device 1 pulls the patient's mandible forward relative to the maxilla, improving the relative positioning of the maxilla and mandible. However, due to habitual movement during treatment, the mandible tends to pull back to its original position, with the pulling force of the maxilla opposing that of the mandible. Therefore, the front side edge 4 of at least the upper bracket 22 includes a protruding front wall 41, and the rear side edge 5 of at least the lower bracket 32 includes a protruding rear wall 51. The front wall 41 is configured to prevent the upper moldable component 21 and/or lower moldable component 31 from being pulled forward and separating from the upper bracket 22 and/or lower bracket 32. The rear wall 51 is configured to prevent the upper moldable component 21 and/or lower moldable component 31 from being pulled backward and separating from the upper bracket 22 and/or lower bracket 32. In some cases, the front wall 41 and/or the rear wall 51 of the upper bracket 22 and/or lower bracket 32 may form grooves to receive the upper moldable component 21 and/or lower moldable component 31, creating a more secure connection between the upper bracket 22 and/or lower bracket 32 and the upper moldable component 21 and/or lower moldable component 31.

The primary operational mechanism of the mandibular advancement device 1 involves engaging the upper moldable component 21 with the upper bracket 22 to form the upper tray assembly 2, and engaging the lower moldable component 31 with the lower bracket 32 to form the lower tray assembly 3. By fixing the relative positions of the upper tray assembly 2 and the lower tray assembly 3, the device pulls the patient's mandible forward, away from the lingual side, to open the patient's airway. Typically, the upper tray assembly 2 and the lower tray assembly 3 are two completely separate parts, and the connection between them is generally achieved by the upper bracket 22 and the lower bracket 32. Specifically, to prevent the separation of components during use and ensure the effectiveness of the treatment, a connection module 7 is provided between the upper bracket 22 and the lower bracket 32. The connection module 7 ensures the proper function of the mandibular advancement device 1 by moving the lower tray assembly 3 forward relative to the upper tray assembly 2, toward the labial side, securing the patient's mandible in the correct and appropriate position. In some special cases, the upper tray assembly 2 and the lower tray assembly 3 may be connected through a hinge or any other mechanism, forming an inseparable unit. In the upper tray assembly 2 and the lower tray assembly 3, the upper moldable component 21 and the lower moldable component 31 may be removably engaged with the upper bracket 22 and the lower bracket 32, or they may be fixed in a non-removable manner. The removable engagement can take the form of snap-fits 72, which consist of protrusions and grooves, and/or additional components such as pins, screws, bolts, or magnets. For better fixation and ease of use of the mandibular advancement device 1, the preferred method of connecting the upper moldable component 21 and lower moldable component 31 to the upper bracket 22 and the lower bracket 32 is a snap-fit 72 connection. In this configuration, the snap-fit 72 typically involves protrusions on the upper moldable component 21 and lower moldable component 31 that engage with corresponding grooves to receive the protrusions on the upper bracket 22 and the lower bracket 32, respectively. Alternatively, the protrusions may be located on the upper bracket 22 and the lower bracket 32, and the grooves to receive the protrusions on the upper moldable component 21 and lower moldable component 31. The protrusions and grooves can be positioned either on both sides near the middle section (i.e., the part of the mandibular advancement device 1 nearer the patient's incisors) and/or symmetrically positioned at both ends (i.e., the part of the device 1 nearer the patient's temporomandibular joint).

The material selection for the upper tray assembly 2 and the lower tray assembly 3 is broad. Since the upper moldable component 21 and the lower moldable component 31 primarily come into contact with the patient's teeth and parts of the gums during use, while the upper bracket 22 and the lower bracket 32 serve as supporting, fixing, and positioning elements within the mandibular advancement device 1, the upper tray assembly 2 and lower tray assembly 3 are made from at least two different materials. Specifically, the hardness of the materials used for the upper bracket 22 and the lower bracket 32 is at least partially greater than the hardness of the materials used for the upper moldable component 21 and the lower moldable component 31. The hardness of the upper bracket 22 and the lower bracket 32 ranges from Shore D45 to Shore D95. In some cases, the upper tray assembly 2 and the lower tray assembly 3 may be made from a single material, with variations in rigidity achieved through specialized manufacturing processes. When selecting materials for the mandibular advancement device 1, several factors are considered, including biocompatibility, durability, functional stability, patient comfort, and ease of cleaning. Therefore, plastics and polymers that are suitable for use in oral medical devices and capable of being molded are chosen. These materials include, but are not limited to, polypropylene (PP), polycarbonate (PC), polyethylene terephthalate (PET), polymethyl methacrylate (PMMA), polyphenylsulfone (PPSU), polyethylene (PE), high-density polyethylene (HDPE), low-density polyethylene (LDPE), ethylene-vinyl acetate (EVA), thermoplastic polyurethane (TPU), styrene-ethylene-butylene-styrene (SEBS), styrene-ethylene-propylene-styrene (SEPS), silicone rubber, and others.

The required temperatures at which the upper moldable component 21 and the upper bracket 22 are heated to deform differ, as do the required temperatures for the lower moldable component 31 and the lower bracket 32. The upper moldable component 21 and the lower moldable component 31 are made of a moldable material, with the temperature required to heat them to deform being less than 100° C. The upper bracket 22 and the lower bracket 32 are made of a material with high thermal stability, requiring a temperature greater than 100° C. to deform. Specifically, the upper moldable component 21 and the lower moldable component 31 include flexible thermoplastic materials and are configured to soften when heated in hot water, allowing them to be adjusted and molded to conform to the shape of the patient's upper and lower dental arches when the patient bites. (In this context, “hot water” refers to water heated to at least 60°C.) During use, the patient simply places the upper tray assembly 2 and lower tray assembly 3 in hot water, allows them to cool slightly, then gently bites down on them to ensure a proper fit with the teeth. This process enables the upper moldable component 21 and lower moldable component 31 to conform to the dental arches through biting. The flexible thermoplastic material provides the necessary flexibility and is capable of withstanding repeated mechanical stress without failure, which is crucial for patients biting down on the moldable components of the mandibular advancement device 1. Additionally, the flexible thermoplastic material is lightweight and environmentally friendly.

The upper bracket 22 and the lower bracket 32 need to be made from materials that are more stable than the upper moldable component 21 and lower moldable component 31. These materials should have high thermal stability and be able to retain their original shape after molding, providing structural support for the mandibular advancement device 1. In most cases, the upper bracket 22 and the lower bracket 32 have to endure prolonged use while maintaining their shape. Therefore, they are made of more durable materials compared to the upper moldable component 21 and lower moldable component 31. In some cases, the upper bracket 22 and the lower bracket 32 are also made of thermoplastic materials, and in such circumstances, the required temperatures at which the upper bracket 22 and the lower bracket 32 are heated to deform will be higher than those of the upper moldable component 21 and lower moldable component 31, ensuring that the upper and lower brackets are not affected during the molding process of the moldable components.

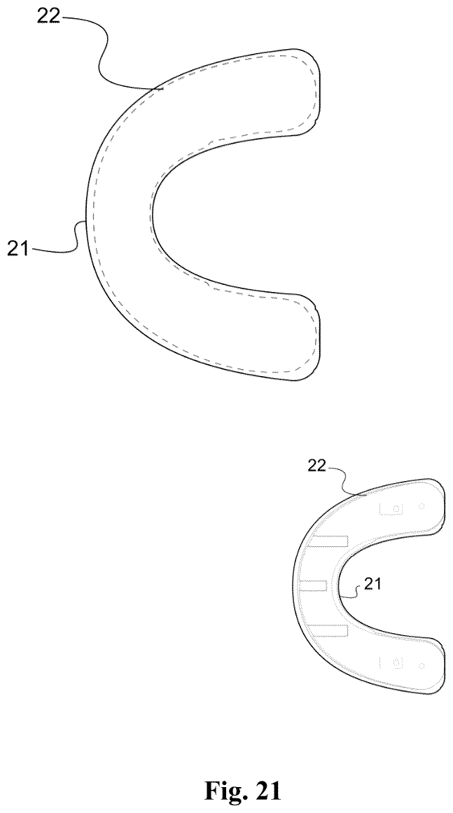

In this disclosure, the dimensions of the upper moldable component 21 and lower moldable component 31, as well as the upper bracket 22 and the lower bracket 32, are specified. The projected area of the upper moldable component 21 on a horizontal plane is greater than that of the upper bracket 22 on the same plane, and the projected area of the lower moldable component 31 on a horizontal plane is greater than that of the lower bracket 32 (as shown in FIG. 21), to provide greater comfort and adjustability. Firstly, the upper moldable component 21 and lower moldable component 31 are made from more flexible materials than the upper bracket 22 and the lower bracket 32, reducing irritation and abrasion to the soft tissues of the oral cavity. The flexible material used for the upper moldable component 21 and lower moldable component 31 weighs at least 3.3 g. Secondly, considering the variation in the distance from the inner side of the lips to the posterior molar area among different patients, a device that is too long may cause irritation to the soft tissues in the posterior molar region, especially in areas near the joints, potentially affecting occlusal function. For patients where the size of the upper moldable component 21 and lower moldable component 31 is unsuitable, the length can be trimmed and adjusted based on personal needs and preferences, enhancing comfort and personalized adaptation. This design not only ensures the functionality of the mandibular advancement device 1 but also allows patients more control over adjustments. Furthermore, since the height of the upper and lower dental arches in the oral cavity tends to be higher in the middle and lower on both sides, the upper tray assembly 2 and lower tray assembly 3 are also designed with a higher middle section and lower sides to prevent irritation and abrasion to the soft tissues of the oral cavity.

Detailed embodiments are presented below to elucidate the configurations of mandibular advancement device 1 provided in this disclosure.

### Embodiment 1

The mandibular advancement device 1 in this embodiment includes: an upper tray assembly 2, configured to be positioned adjacent to the upper dental arch of a patient when placed inside the mouth; and a lower tray assembly 3, configured to be positioned adjacent to the lower dental arch of the patient when placed inside the mouth. The upper tray assembly 2 includes an upper moldable component 21 and an upper bracket 22, and the required temperatures at which the upper moldable component 21 and the upper bracket 22 are heated to deform differ. The lower tray assembly 3 includes a lower moldable component 31 and a lower bracket 32, and the required temperatures at which the lower moldable component 31 and the lower bracket 32 are heated to deform differ.

The upper bracket 22 and the lower bracket 32 each have a front side edge 4 near the labial side and a rear side edge 5 near the lingual side. At least the upper bracket 22 has a protruding front wall 41 on the front side edge 4, and at least the lower bracket 32 has a protruding rear wall 51 on the rear side edge 5.

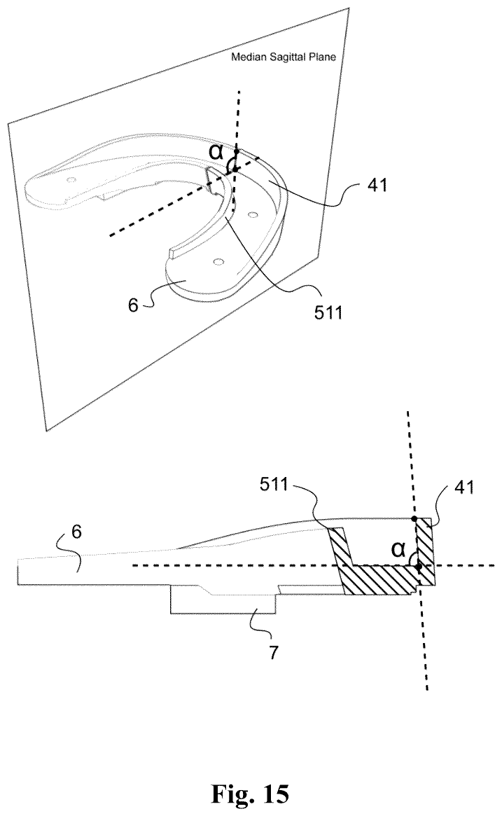

The rear wall 51 of at least the lower bracket 32 includes a baffle section 511 at the median sagittal plane. At least a portion of the front wall 41 of at least the upper bracket 22 is inclined towards the lingual side (as shown in FIG. 15).

The front wall 41 and the rear wall 51 of at least the upper bracket 22 include a clamping section 512 that is positioned near the molar area when placed inside the patient's mouth. The rear wall 51 of at least the lower bracket 32 also includes a baffle section 511 at the median sagittal plane.

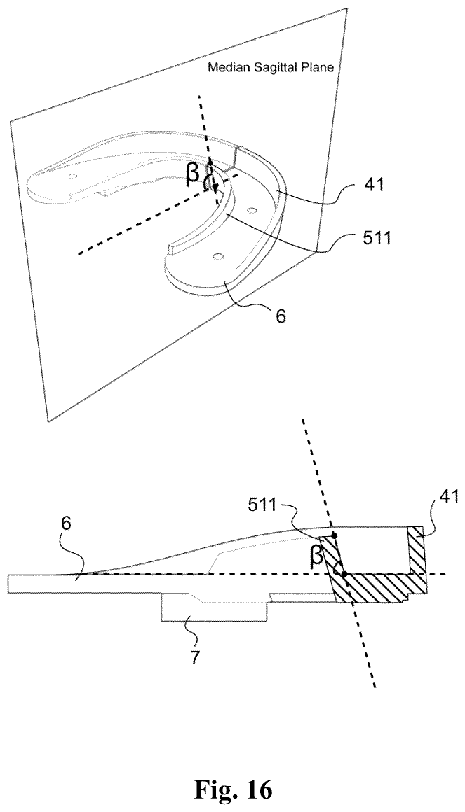

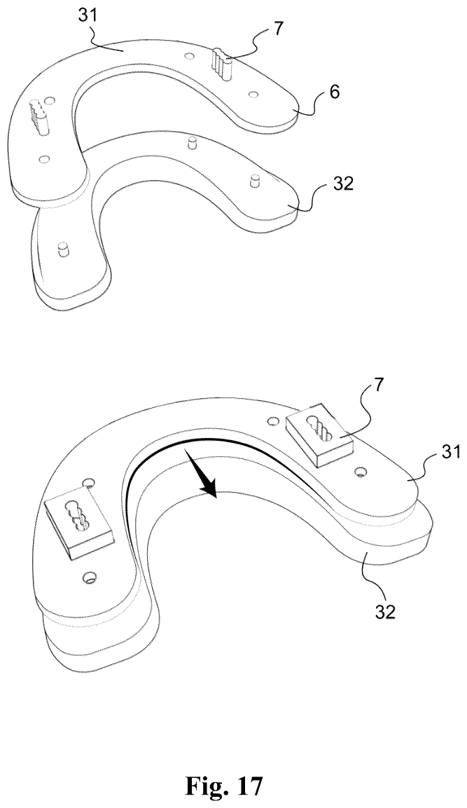

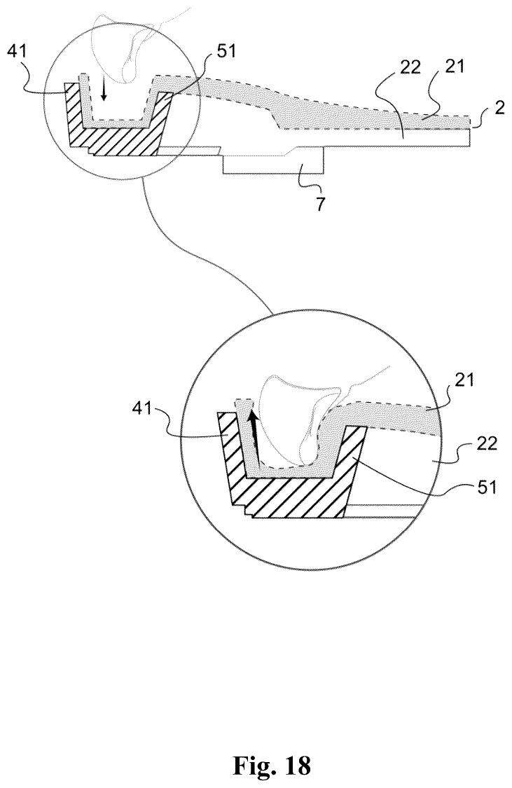

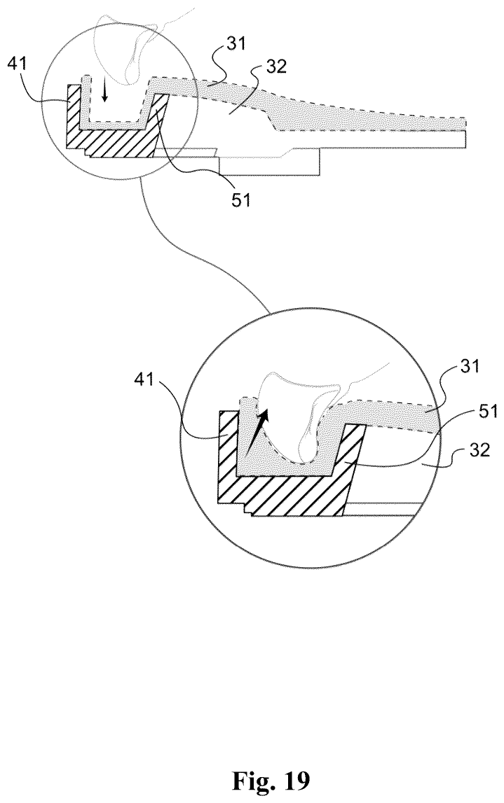

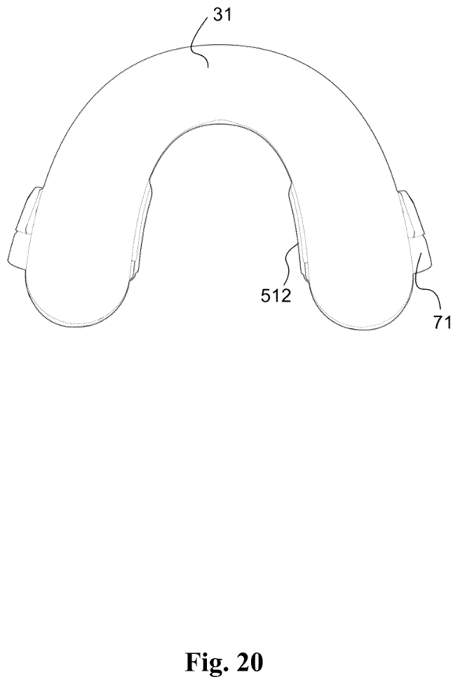

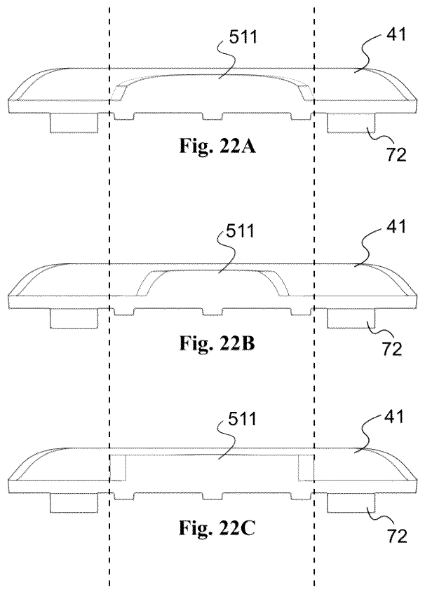

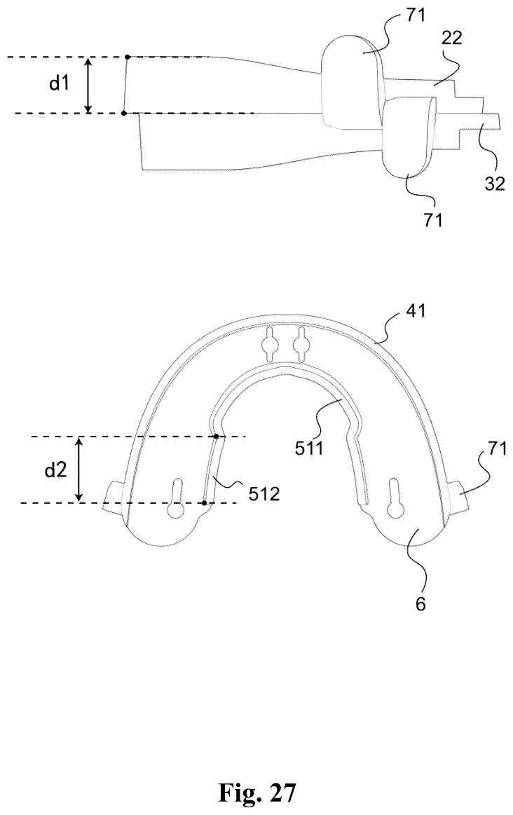

The opening and closing of the mouth primarily relies on the rotation and sliding of the mandible at the temporomandibular joint. When the condyle of the mandible rotates within the glenoid fossa, the mandible moves upward and forward, thereby closing the mouth. Therefore, when the patient bites down on the mandibular advancement device 1 during the molding process, it is the mandible that pushes the lower tray assembly 3 upward, pressing the lower tray assembly 3 against the upper tray assembly 2, which is in contact with the upper dental arch, to complete the molding process. However, the outer surface of the central incisors in the upper dental arch is often angled relative to the vertical plane. As a result, when the upper tray assembly 2 presses the upper moldable component 21 against the upper dental arch, it creates a gap between the vertical inner surface of the molded upper moldable component 21 and the inwardly inclined outer surface of the central incisors, preventing a proper fit (as shown in FIG. 18). This gap can cause the upper tray assembly 2 to detach from the upper dental arch during wear, which undermines the effectiveness of the treatment. Therefore, the upper bracket 22 of the mandibular advancement device 1 has been modified to address this issue. The front wall 41 of at least the upper bracket 22 is designed with at least a portion inclined towards the lingual side. This improvement ensures that, during the biting process, the inclined front wall 41 of at least the upper bracket 22 directs the overflowing material of the upper moldable component 21 towards the lingual side, filling or narrowing the gap that would otherwise form, thereby enhancing the stability of the mandibular advancement device 1 (as shown in FIG. 19). The angle of inclination of the front wall 41 determines the stability and comfort of the fit between at least the upper tray assembly 2 and at least the upper dental arch. If the inclination angle is too steep, it could cause discomfort or gum irritation for the patient, while an angle that is too shallow may compromise the effectiveness of the design. Thus, the front wall 41 of at least the upper bracket 22 is designed with an outer wall facing the labial side and an inner wall opposite it. The angle between the inner wall of the front wall 41 of at least the upper bracket at a median sagittal plane and the horizontal plane is less than 90° (as shown in FIG. 15, ∠α≤90°). Based on extensive research into the oral dimensions of various patient groups, the height of the front wall 41 of at least the upper bracket 22 is designed to provide optimal functionality while ensuring comfort within the patient's mouth. Therefore, the height of the front wall 41 of at least the upper bracket 22 is set to be less than 19.5 mm (as shown in FIG. 27, d1≤19.5 mm). The primary function of the mandibular advancement device 1 is to pull the patient's mandible forward (away from the lingual side). However, due to habitual movement, the patient's mandible exerts a backward force (towards the lingual side) on the lower moldable component 31. Since the lower moldable component 31 is made of flexible material, this can easily cause excessive deformation or result in misalignment or separation between the lower moldable component 31 and the lower bracket 32 (as shown in FIG. 17). To prevent at least the lower moldable component 31 and at least the lower bracket 32 from sliding or detaching during wear, the rear wall 51 of at least the lower bracket 32 includes a baffle section 511 at the median sagittal plane. The baffle section 511 helps provide resistance against deformation forces acting on the lower moldable component 31, maintaining its original shape and securing the connection between at least the lower moldable component 31 and at least the lower bracket 32. This prevents detachment due to habitual use during wear. This design directly impacts the treatment efficacy of the mandibular advancement device 1. The device 1 with the baffle section 511 is more effective in alleviating the symptoms of obstructive sleep apnea syndrome. In some cases, at least the lower moldable component 31 has a shape that engages and matches with the baffle section 511 to optimize the size and comfort of the mandibular advancement device 1. Specifically, the lower moldable component 31 fits tightly against the baffle section 511, ensuring that the size of the lower tray assembly 3 does not increase due to the addition of the baffle section 511. This tight-fitting design not only ensures the stability of the components when positioned inside the patient's mouth but also effectively reduces the sensation of a foreign object in the mouth. The baffle section 511 can be provided either on the upper bracket 22 or the lower bracket 32 individually. The preferred embodiment is to have it on the lower bracket 32. However, the baffle section 511 can also be configured on both the upper bracket 22 and the lower bracket 32 simultaneously. To ensure the functionality of the baffle section 511, its parameters are defined accordingly in this embodiment. The length of the baffle section that is provided at the rear side edge is greater than 10% of the total length of the rear side edge of the lower bracket. Additionally, the baffle section 511 positioned at the central portion of at least the lower bracket 32 is considered the optimal arrangement. Since the overall shape of the upper bracket 22 and the lower bracket 32 is arcuate, overly extended baffle sections on both sides would be ineffective. Based on data collected from various patient groups regarding oral dimensions, the preferred length of the baffle section 511 is greater than or equal to 5 mm. Due to the limitations in material and size, the thickness of the baffle section 511 is set in the range of 0.3 mm to 10 mm, providing adequate support to the moldable components. In most cases, the baffle section 511 will not extend beyond the end of the patient's gums. Therefore, at least in the lower tray assembly 3, the maximum height of the baffle section 511 is less than or equal to the maximum height of the lower moldable component 31, and the height of the baffle section 511 is set to be less than or equal to 15 mm. The baffle section 511 may take various forms: it can be designed with a higher center and lower sides to conform to the structure of most patients'oral cavity, or it can have a flat top end. The top and bottom edges of the baffle section 511 may have continuous curvature or discontinuous curvature. The length of the baffle section 511 can also be adjusted based on practical needs, taking either a longer or shorter form (as shown in FIGS. 22A, 22B and 22C). To better conform to the oral s structure, the baffle section 511 of the rear wall 51 may be designed with an inclined angle. The angle between the inner wall of the baffle section 511 at the median sagittal plane and the horizontal plane is set within the range of 20° to 150°, with a preferred range of 45° to 90° (as shown in FIGS. 16, 20°≤∠β≤150°).

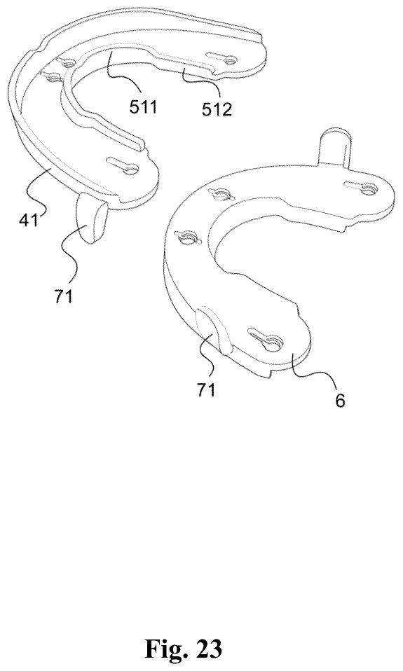

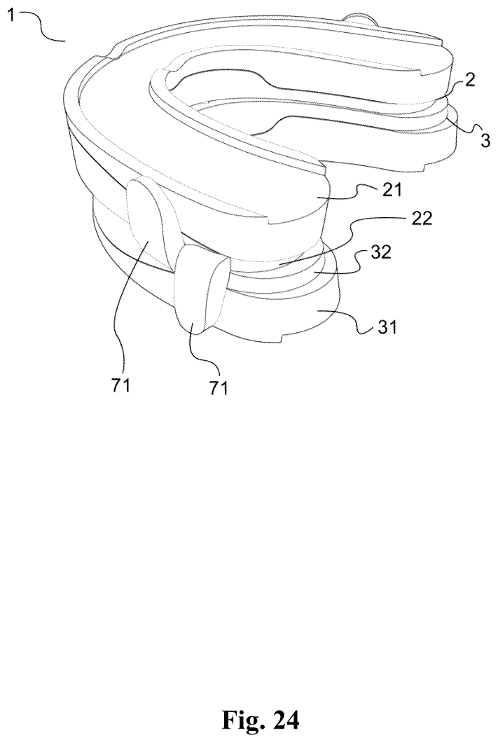

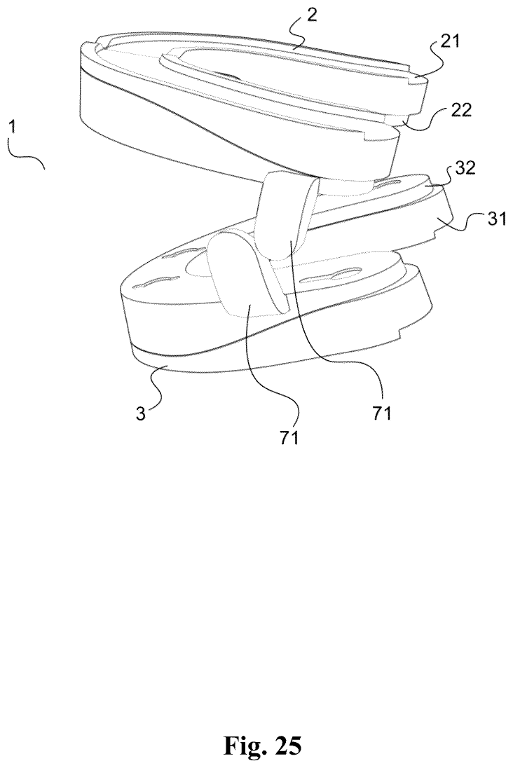

The connection module 7 that is provided between the upper bracket 22 and the lower bracket 32 includes a protrusion 71 provided on the front wall 41 and/or the rear wall 51 of the upper bracket 22 and/or the lower bracket 32. The inclusion of the protrusion 71 allows the patient to open and close their mouth during treatment (as shown in FIGS. 23-25), which enhances the flexibility of the mandibular advancement device 1 and improves patient compliance compared to traditional connection modules. The function of the protrusion 71 relies on its height. By positioning the protrusion 71 on the upper tray assembly 2 and lower tray assembly 3, it controls the front-to-back alignment of the two assemblies. Even when the upper tray assembly 2 and the lower tray assembly 3 are separated, the length of the protrusion 71 ensures that once they come into contact, the protrusions limit their movement, maintaining the positional relationship between the two assemblies. This allows the mandibular advancement device 1 to function effectively while still enabling the patient to open and close their mouth (as shown in FIG. 24, where the upper tray assembly 2 and lower tray assembly 3 are in a closed configuration, and in FIG. 25, where they are in an open configuration. In both cases, the mandibular advancement function is still achieved). The protrusion 71 is generally positioned to connect with the front wall 41 of the upper bracket 22 and/or the lower bracket 32. In some special cases, the protrusion 71 may also be positioned to connect with the rear wall 51 of the upper bracket 22 and/or the lower bracket 32. The protrusion 71 can take various forms. In one embodiment, both the upper bracket 22 and the lower bracket 32 of the mandibular advancement device 1 have a protrusion 71, or the protrusion may be provided on only one of the brackets-either the upper bracket 22 or the lower bracket 32.

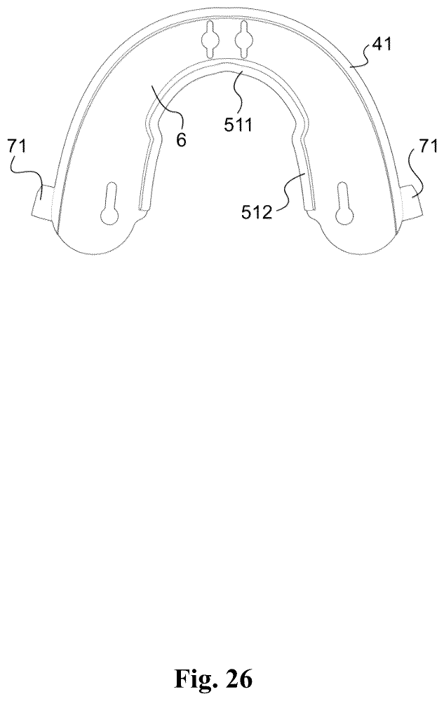

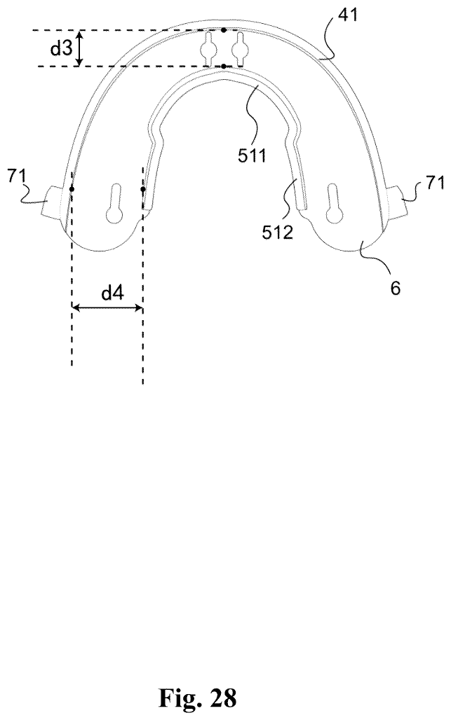

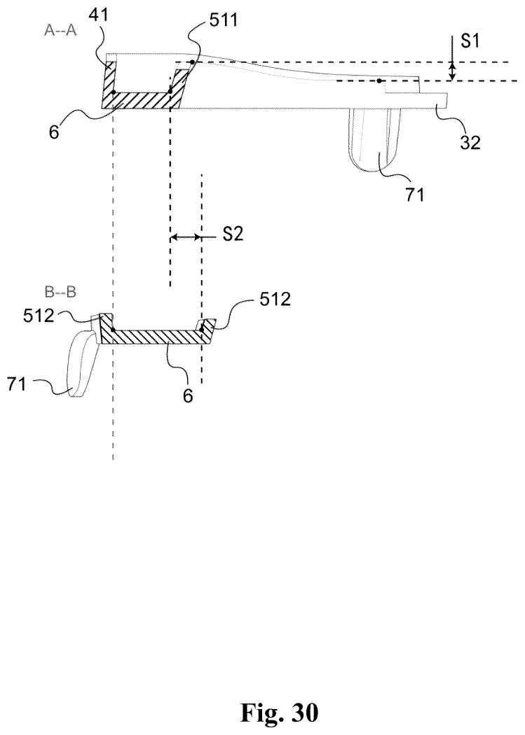

The front wall 41 of the front side edge 4 and the rear wall 51 of the rear side edge 5 of the upper bracket 22 and the lower bracket 32 extend along the front side edge 4 and rear side edge 5, respectively, and include clamping sections 512 (as shown in FIG. 26). In the upper and lower dental arches, molars are typically wider and shorter than other teeth, making them more difficult to engage and clamp with the upper moldable component 21 and lower moldable component 31. This increases the likelihood of the upper tray assembly 2 and lower tray assembly 3 separating from the upper and lower dental arches, respectively, especially since the upper tray assembly 2 tends to separate from the upper dental arch under the influence of its own weight. Through the analysis of molar data and after multiple design attempts and tests, it was concluded that adding clamping sections 512 to the side walls of the upper bracket 22 and the lower bracket 32 at the points of contact with the molars when positioned in the patient's mouth is an effective solution. The clamping sections 512 on the front wall 41 and/or rear wall 51 can prevent the softened material of the upper moldable component 21 and/or lower moldable component 31 from spreading to the sides during the patient's bite. The upper tray assembly 2 and/or lower tray assembly 3 with clamping sections 512 force the softened material to tightly clamp around the molar area during the bite, increasing the contact surface area between the molded upper moldable component 21 and/or lower moldable component 31 and the molars. The design of the clamping sections 512 ensures a secure connection between the key regions of the upper bracket 22 and the lower bracket 32 and the molars. This improvement enables the upper tray assembly 2 and/or lower tray assembly 3 to better maintain their original positions during wear, reducing the likelihood of detachment and enhancing the stability and reliability of the mandibular advancement device 1. Further data parameters were defined to improve its functionality. Through research and data collection on the length and width of molars across various populations and even different ethnic groups, dimensions for the clamping section were determined, such as length, width, and height, that best suit a broad range of users. The goal is to minimize the impact on patients while ensuring the clamping section performs effectively. The length of the clamping section is set to be greater than or equal to 20 mm (as shown in FIG. 27, d2). Since molars are naturally wider and lower compared to other teeth, the distance between the front wall 41 and rear wall 51 at the clamping section 512 is greater than in other areas, and the height is lower at the clamping section 512 (as shown in FIG. 30, with height difference hl and width difference h2 between the clamping section 512 and other areas). Therefore, at least one shortest distance between the front wall 41 and rear wall 51 at the clamping section 512 is greater than the corresponding shortest distance between the front wall 41 and rear wall 51 at other areas of the upper bracket 22 and the lower bracket 32, with the ratio of the two distances being at least 1.12:1 (as shown in FIG. 28, d4 to d3 ratio). The walls of the clamping section 512 may be angled with respect to the bottom wall 6, or they may be perpendicular to the bottom wall 6.

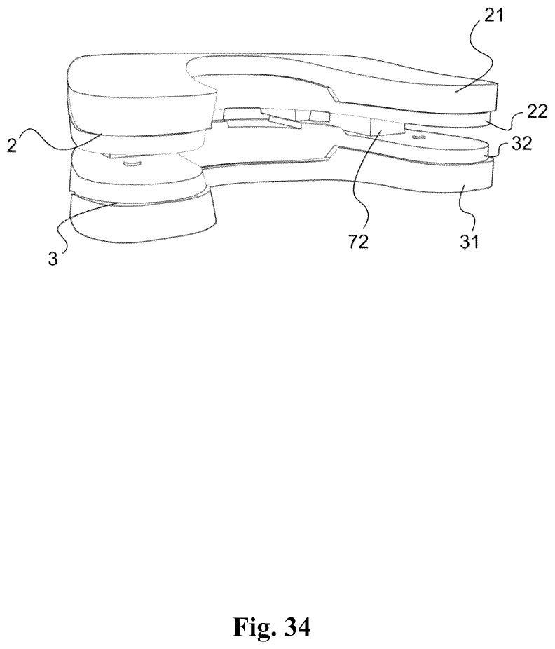

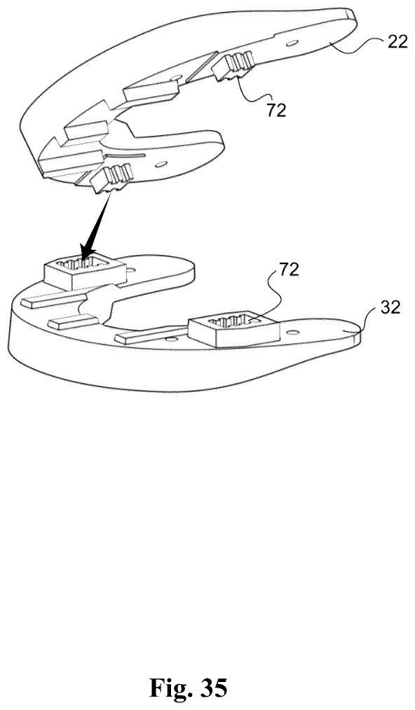

In addition to the protrusion, the connection module 7 can also take other forms that function similarly, although differing in structure. Specifically, both the upper bracket 22 and the lower bracket 32 have a bottom wall 6 that is positioned opposite the occlusal surface of the teeth when the patient bites. The connection module 7 includes a position adjustment snap-fit 72 provided on the bottom wall 6 (as shown in FIGS. 34 and 35). In this configuration of the mandibular advancement device 1, the upper tray assembly 2 and lower tray assembly 3 cannot be separated once positioned in the patient's mouth, meaning the patient cannot open or close their mouth while wearing the device. In this embodiment of the mandibular advancement device 1, the position adjustment snap-fit 72 can have multiple (two or more) adjustable positions, allowing the patient to select the most comfortable position. The position adjustment snap-fit 72 can take various forms and is generally located on the upper bracket 22 and the lower bracket 32 nearer the lingual side. However, in some special cases, the position adjustment snap-fit 72 may be positioned elsewhere. The position adjustment snap-fit 72 may be a protrusion on the upper bracket 22 that engages with a corresponding groove on the lower bracket 32, or a protrusion on the lower bracket 32 that engages with a groove on the upper bracket 22. In some cases, the position adjustment may take forms other than protrusions, grooves, or snap-fits. Additionally, there may be multiple fixed connections between the upper bracket 22 and the lower bracket 32, apart from the position adjustment snap-fit 72, providing extra security, or the position adjustment snap-fit 72 alone may be responsible for securing the upper bracket 22 and the lower bracket 32.

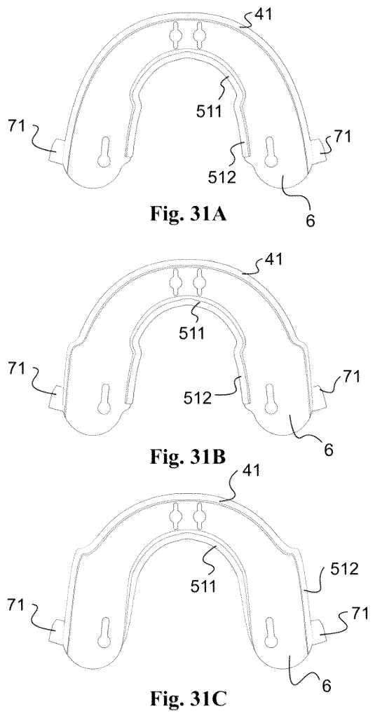

In other embodiments, the upper bracket 22 and/or lower bracket 32 may have different shapes in the clamping section 512 to accommodate various molar configurations, allowing for adaptation to different populations with longer, wider, or shorter molars (as shown in FIGS. 31A, 31B and 31C).

In other embodiments, the connection module 7 may take various forms beyond the protrusion 71 and position adjustment snap-fit 72. These forms include, but are not limited to, threaded connections, hook connections, piston connections, welded connections, plug-in connections, pin connections, adhesive connections, spring connections, mortise and tenon connections, or elastic band connections. The connection method can employ one form or a combination of two or more forms. The selection of the appropriate connection method depends on the specific design requirements, material properties, and usage conditions, ensuring a secure, reliable, and durable connection while also being easy to assemble during production and simple for the patient to adjust.

### Embodiment 2

In this embodiment, the mandibular advancement device 1 includes: an upper tray assembly 2, having an arcuate outer shape to conform to the curve of the patient's upper dental arch; and a lower tray assembly 3, having an arcuate outer shape to conform to the curve of the patient's lower dental arch. The upper tray assembly 2 includes an upper moldable component 21 and an upper bracket 22, with the required temperatures at which the upper moldable component 21 and the upper bracket 22 are heated to deform being different. The lower tray assembly 3 includes a lower moldable component 31 and a lower bracket 32, with the required temperatures at which the lower moldable component 31 and the lower bracket 32 are heated to deform being different.

The upper bracket 22 and the lower bracket 32 each have a front side edge 4 near the labial side and a rear side edge 5 near the lingual side. At least the upper bracket 22 has a protruding front wall 41 on the front side edge 4, and at least the lower bracket 32 has a protruding rear wall 51 on the rear side edge 5.

The front wall 41 of the upper bracket 22 and the lower bracket 32, and the rear wall 51 of the upper bracket 22 and the lower bracket 32, include a clamping section 512 positioned near the molar area when placed inside the patient's mouth. Furthermore, at least the rear wall 51 of the lower bracket 32 includes a baffle section 511 at the median sagittal plane.

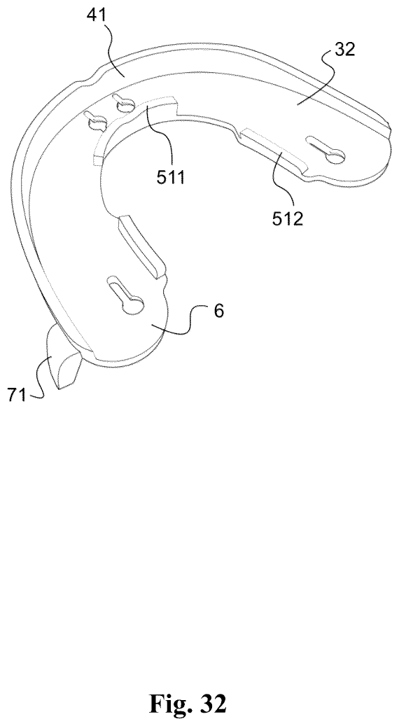

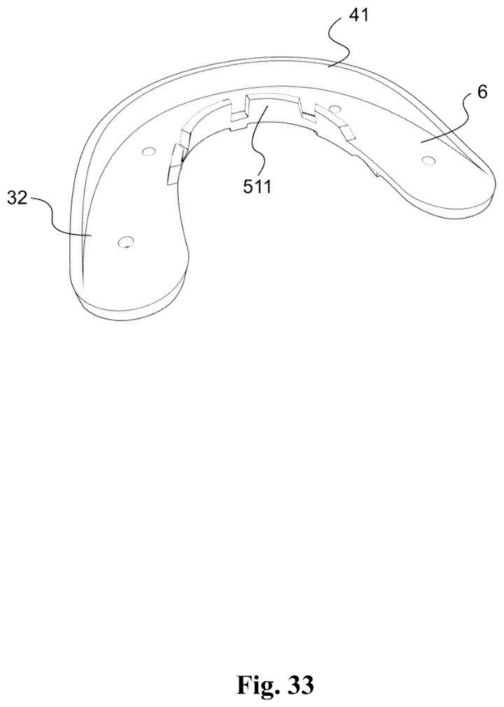

The difference between this embodiment and Embodiment 1 lies in that at least one of the front wall 41 of the upper bracket 22 or lower bracket 32 or the rear wall 51 of the upper bracket 22 or lower bracket 32 is in a discontinuous form, composed of multiple separated walls with gaps, while retaining the same functionality. There are two configurations of this discontinuous form: In the first configuration, different functional sections of the front wall 41 and rear wall 51 of the upper bracket 22 and/or lower bracket 32 are separated, such as the baffle section 511 and the clamping section 512, which are distinct and disconnected (non-integrated walls) (as shown in FIG. 32). In the second configuration, the wall serving the same function is segmented, such as multiple separated walls forming the baffle section 511 (as shown in FIG. 33). This discontinuous wall design reduces the amount of material used in the upper bracket 22 and/or lower bracket 32 of the mandibular advancement device 1, achieving a lightweight structure, and improving the overall comfort for the patient.

On the other hand, the discontinuous walls increase the flexibility of the front wall 41 and/or the rear wall 51 of the upper bracket 22 and/or lower bracket 32 to a certain extent. This added flexibility allows patients who may not have been perfectly suited to the original design of the upper bracket 22 and/or lower bracket 32 to adjust the shape of the device slightly within their mouths, better accommodating the movement and shape of their mandibles and reducing the pressure on their teeth. For instance, if the angle of the front wall 41 and/or rear wall 51 of the upper bracket 22 and/or lower bracket 32 is not initially aligned with the patient's optimal comfort, the segmented design allows these walls to be more easily adjusted to fit the patient's unique oral structure, providing a more personalized fit. This flexibility ensures that each patient can adjust the device to their ideal angle and size, improving both comfort and the effectiveness of the treatment. Moreover, since the front wall 41 and rear wall 51 of the upper bracket 22 and lower bracket 32 include multiple functional sections, separating these walls ensures that the various functions of these sections can be achieved independently without interference. The structure of the discontinuous walls also provides additional benefits, such as improving the breathability of the device, reducing the sense of heat and discomfort during use.

Additionally, the technical features described in the above embodiments can be combined as needed to obtain a mandibular advancement device 1 that includes all or part of these features.

1. Through comprehensive research, design, and experimentation, this disclosure introduces a novel mandibular advancement device that aligns with the oral structure of the human body while further enhancing its effectiveness and stability. Initial analysis of the current market revealed several areas where existing mandibular advancement devices need improvement, particularly with non-custom-made devices, which frequently exhibit the problem of falling out when the mouth opens. Feedback collected from various e-commerce platforms showed that non-custom-made mandibular advancement devices generally received poor reviews. Following a detailed assessment of patient needs and an extensive analysis of oral structure data, several key factors contributing to these issues were identified across different patient groups'oral structures and usage habits: a. The upper tray assembly tends to fall downward under the influence of gravity; b. The outer surface of the patient's central incisors does not fit well with the moldable component; c. The upper and lower tray assemblies do not adequately conform to the patient's molars, posing a risk of dislodgement; d. when the patient bites, the lower moldable component is susceptible to forces exerted by the mandible returning to its original position, causing it to separate from the lower bracket and rendering the device ineffective. Each of these issues was analyzed to identify the underlying causes, and corresponding solutions were developed. Extensive testing was then conducted, leading to the development of a more effective mandibular advancement device grounded in both theory and practice. (1) The occlusion between the upper and lower dental arches is essentially the rotation and movement of the mandible in the glenoid fossa around the condyle, causing the mandible to move towards the maxilla. Therefore, during the shaping process of the upper moldable component, the lower dental arch pushes both the upper and lower tray assemblies upward until the upper moldable component compresses against the upper dental arch. However, because the outer surface of the central incisors is slanted rather than vertical, and the upper moldable component is compressed upward vertically, the surface it presses against becomes a vertical inner surface. This difference in angles creates a gap, preventing the upper moldable component from properly fitting the upper dental arch, which leads to the upper tray assembly falling out. To address this, the design includes a front wall with an inward inclination, which pushes the overflowing material towards the outer surface of the central incisors during the upward compression of the upper tray assembly. This ensures that the upper moldable component conforms perfectly to the upper dental arch during the shaping process. The inwardly inclined front wall also works in conjunction with the rear wall to securely hold the central incisors. The combination of the inclined front and rear walls provides a more secure clamp on the central incisors, ensuring the overall stability of the mandibular advancement device. (2) Molars are relatively short, and their contact surface with the upper moldable component and the lower moldable component is insufficient, which, combined with the weight of the upper tray assembly, often causes the mandibular advancement device to fall out at the molar region. To improve the stability of the upper and lower tray assemblies when placed inside the patient's mouth, at least the upper bracket includes a front wall and a rear wall positioned to contact the patient's teeth. Nearer the molar areas, the front wall and the rear wall form a clamping section that, during the molding process of the upper moldable component and the lower moldable component when the patient bites down, helps direct the overflowing material upwards, increasing the contact surface area between the moldable components and the upper and lower dental arches. Additionally, the clamping section helps secure the molars, making the connection between the upper and lower tray assemblies more stable. (3) The working principle of the mandibular advancement device involves fixing the relative positions of the upper and lower tray assemblies to control the relative positioning of the upper and lower dental arches for corrective purposes. Although the lower tray assembly moves the lower dental arch forward, this movement opposes the natural position of the mandible, causing a backward force to act on the lower tray assembly during use. This force typically acts on the lower moldable component. If the connection between the lower moldable component and the lower bracket is not secure, the lower moldable component may shift relative to the lower bracket, leading to device failure. To address this, a rear wall is set at the median sagittal plane of at least the lower bracket, further forming what is referred to as a baffle section. The baffle section provides resistance against the dislodging force acting on the lower moldable component, maintaining its fixed position relative to the lower bracket and ensuring the device remains effective. (4) After incorporating the above structures to enhance stability, repeated experimental testing and multiple iterative optimizations were carried out to mitigate the influence of gravity on the upper tray assembly and enhance wearing comfort. This refined structure allows the device to maximize its effectiveness while ensuring patient comfort during use.

2. Dentistry is a precise and data-driven field, where even small variations in data-such as angle, weight, height, width, and material flexibility-can affect patient comfort and device effectiveness. This disclosure carefully fine-tunes these parameters through repeated testing to achieve optimal performance and better comfort for patients. By refining the design structure and conducting detailed data analysis and calibration, the disclosure results in a more comfortable mandibular advancement device, which improves patient treatment adherence. (1) the upper bracket and the lower bracket are designed to be wider at the clamping section compared to other areas. This improvement is based on the fact that molars are typically wider than other teeth in the oral structure, yet existing mandibular advancement devices do not adjust the width of the clamping section accordingly. The widened clamping section allows the upper moldable component and the lower moldable component to better conform to the patient's upper and lower dental arches after molding, ensuring a secure but not overly tight clamp on the molars, enhancing comfort. (2) The baffle section of at least the lower bracket is designed with an angle that aligns with the natural shape of the gums and teeth. Since the gums and lower teeth generally do not have vertical surfaces but have sloped ones, the angled baffle section matches the slope of the gums and teeth. This design allows the mandibular advancement device to fit more snugly within the patient's mouth, enhancing comfort during use. Comfort is a critical factor for patients—if the device causes discomfort or issues such as component dislodgement during use, patients may be less willing to wear it for extended periods, which could negatively impact treatment outcomes. By improving comfort, this disclosure increases patient adherence and contributes to more stable and effective treatment. (3) Additionally, this disclosure incorporates specific parameter adjustments to improve the effectiveness of the mandibular advancement device during use. For example, the height of the front wall is limited to ensure that it can securely hold and press against the upper and/or lower dental arches without scraping the gums. The angle of the front wall is also specified to allow the front wall of at least the upper bracket to tilt the upper moldable component towards the lingual side when the patient bites, without clamping the upper dental arch too tightly and causing discomfort. The length of the baffle section is controlled to ensure it provides effective support and stabilization.

3. The addition of the baffle section extends the lifespan of the mandibular advancement device, providing cost-saving benefits for the patient. Devices without a baffle section often face issues like the lower moldable component becoming dislodged, which can easily lead to damage since the upper moldable component and the lower moldable component are made from flexible materials. This increases the frequency and cost of replacing these parts for the patient. Damage not only shortens the device's lifespan but also undermines its stability, weakening its therapeutic effectiveness. In contrast, the mandibular advancement device in this disclosure features a baffle section that strengthens the connection between the lower moldable component and the lower bracket. This design effectively prevents the force from pulling the lower moldable component out of place, ensuring that the device remains securely in place during use and reducing the risk of damage. As a result, the device's stability and durability are improved while maintaining a reliable and effective treatment.

4. Typically, while custom-made mandibular advancement devices offer a higher degree of fit with the teeth and allow for breathing through the mouth, they require technologies like oral scanning for customization. This process incurs significant labor and time costs, and a set of custom-made mandibular advancement devices generally costs patients thousands of dollars. In comparison, the mandibular advancement device provided by this disclosure has several advantages over custom-made devices. a. The mandibular advancement device in this disclosure provides the same benefits as custom-made devices, such as allowing mouth-breathing and reducing the risk of dislodgement. Through features like an inclined front wall, the inclusion of a baffle section, and clamping sections, along with precise structural parameters, this design improves the treatment's effectiveness while ensuring stability throughout the patient's therapy. b. The mandibular advancement device of this disclosure is more cost-effective than custom-made devices, reducing the financial burden on patients. This allows patients with limited budgets to access a high-quality mandibular advancement device. While custom-made devices often require precise instruments and labor, driving up costs, the design and production of this disclosure enable patients to achieve the effects of a custom-made device at a lower cost, making it a more practical and economical choice for certain patient groups. c. The mandibular advancement device of this disclosure can be produced and used quickly, saving patients the wait time required for custom-made devices. This makes it a better option for patients seeking prompt treatment. d. In the market, only custom-made mandibular advancement devices typically offer the ability to open and close the mouth during use. However, the non-custom mandibular advancement device of this disclosure achieves the same functionality, meeting a wider range of patient needs. For example, patients who habitually sleep with their mouths open or those who are prone to snoring during sleep benefit from the device's ability to accommodate mouth opening, offering unique advantages. It not only provides greater comfort but also attracts a broader consumer base. e. The main body of custom-made mandibular advancement devices (i.e., the part in direct contact with the teeth) is typically made of rigid, one-piece material. In contrast, the mandibular advancement device of this disclosure uses flexible materials in the sections that contact the inside of the patient's mouth. This prevents irritation to the oral tissues, reducing the sensation of a foreign object while wearing the device. Additionally, in the event of accidental impacts, the flexible material acts as a buffer, protecting the teeth. This makes the device a safer option compared to traditional designs.

The technical features of the above embodiments can be freely combined. For brevity, not all possible combinations of these features are described here. However, as long as the combinations do not introduce contradictions, they should be considered within the scope outlined by this disclosure.

The embodiments described above represent only a few of the possible implementations of the disclosure. Although the descriptions are specific and detailed, they should not be understood as limiting the scope of this disclosure. It should be noted that for one skilled in the art, various modifications and improvements can be made without departing from the concept of the disclosure, and these are also considered within the scope of protection of this disclosure. Therefore, the scope of protection for this disclosure should be determined by the appended claims.

It must be noted that as used herein and in the appended claims, the singular forms “a”, “an”, and “the” include their plural equivalents, unless the context clearly dictates otherwise.

1. A mandibular advancement device with enhanced stability, comprising:
an upper tray assembly, configured to be positioned adjacent to an upper dental arch of a patient when placed inside a mouth of the patient;
a lower tray assembly, configured to be positioned adjacent to a lower dental arch of the patient when placed inside the mouth of the patient;
wherein the upper tray assembly includes an upper moldable component and an upper bracket, wherein required temperatures at which the upper moldable component and the upper bracket are heated to deform differ;
wherein the lower tray assembly includes a lower moldable component and a lower bracket, wherein required temperatures at which the lower moldable component and the lower bracket are heated to deform differ;
wherein the upper bracket and the lower bracket each have a front side edge configured to be near a labial side of the mouth of the patient and a rear side edge configured to be near a lingual side of the mouth of the patient, at least the upper bracket having a protruding front wall on the front side edge and a protruding rear wall on the rear side edge, and at least the lower bracket having a protruding rear wall on the rear side edge;
wherein the protruding rear wall of at least the lower bracket includes a baffle section at a median sagittal plane;
wherein at least a portion of the protruding front wall of at least the upper bracket is configured to be inclined towards the lingual side at an angle of incline;
wherein the angle of incline extends between an inner wall of the protruding front wall of at least the upper bracket at a medium sagittal plane and a horizontal plane and is less than 90°;
wherein the protruding front wall and the protruding rear wall of the upper bracket each comprises a clamping section formed by the respective protruding front wall and the protruding rear wall, the clamping section being configured to be positioned adjacent to a molar region when placed inside the mouth of the patient.

2. The mandibular advancement device according to claim 1, wherein the upper moldable component and the lower moldable component are configured to soften when heated in hot water to allow them to be adjusted and molded to match shapes of the upper dental arch and the lower dental arch of the patient respectively when the patient bites.

3. The mandibular advancement device according to claim 2, wherein the upper moldable component and the lower moldable component include a flexible thermoplastic material.

4. The mandibular advancement device according to claim 1, wherein a hardness of a material of the upper bracket is at least partially greater than a hardness of a material of the upper moldable component, and a hardness of a material of the lower bracket is at least partially greater than a hardness of a material of the lower moldable component.

5. The mandibular advancement device according to claim 1, wherein the upper bracket and the lower bracket each have a bottom wall that is configured to be opposite an occlusal surface of teeth when the patient bites, and the bottom wall of the upper bracket and the bottom wall of the lower bracket are at least partially in contact when placed inside the mouth of the patient.

6. The mandibular advancement device according to claim 1, wherein a length of the baffle section of the protruding rear wall of the lower bracket is greater than 10% of a total length of the rear side edge of the lower bracket.

7-22. (canceled)

23. A mandibular advancement device with enhanced stability, comprising:
an upper tray assembly, configured to be positioned adjacent to an upper dental arch of a patient when placed inside a mouth of the patient;
a lower tray assembly, configured to be positioned adjacent to a lower dental arch of the patient when placed inside the mouth of the patient;
wherein the upper tray assembly includes an upper moldable component and an upper bracket, wherein required temperatures at which the upper moldable component and the upper bracket are heated to deform differ;
wherein the lower tray assembly includes a lower moldable component and a lower bracket, wherein required temperatures at which the lower moldable component and the lower bracket are heated to deform differ;
wherein the upper bracket and the lower bracket each have a front side edge configured to be near a labial side of the mouth of the patient and a rear side edge configured to be near a lingual side of the mouth of the patient, at least the upper bracket having a protruding front wall on the front side edge and a protruding rear wall on the rear side edge, and at least the lower bracket having a protruding rear wall on the rear side edge;
wherein each of the protruding front wall and the protruding rear wall of at least the upper bracket include a clamping section formed by the protruding front wall and the protruding rear wall, respectively, and the clamping section being configured to be positioned near a molar area when placed inside the mouth of the patient;
wherein the protruding rear wall of at least the lower bracket further includes a baffle section at a median sagittal plane;
wherein, at a median sagittal plane, an inner wall of the protruding front wall of at least the upper bracket forms a lingually-inclined angle of less than 90° with a horizontal plane.

24. The mandibular advancement device according to claim 23, wherein the upper tray assembly and the lower tray assembly have an arcuate outer shape.

25. The mandibular advancement device according to claim 23, wherein the baffle section is divided into multiple spaced-apart sections.

26. The mandibular advancement device according to claim 23, wherein at least the lower moldable component has a shape configured to engage and match with the baffle section.

27. The mandibular advancement device according to claim 23, wherein the upper moldable component and the lower moldable component are made of a moldable material, and a temperature required to heat them to deform is less than 100 degrees Celsius.

28. The mandibular advancement device according to claim 27, wherein the upper bracket and the lower bracket are made of a material with high thermal stability, and a temperature required to heat them to deform is greater than 100 degrees Celsius.

29. A mandibular advancement device with enhanced stability, comprising:
an upper tray assembly, configured to be positioned adjacent to an upper dental arch of a patient when placed inside a mouth of the patient;
a lower tray assembly, configured to be positioned adjacent to a lower dental arch of the patient when placed inside the mouth of the patient;
wherein the upper tray assembly includes an upper moldable component and an upper bracket, wherein required temperatures at which the upper moldable component and the upper bracket are heated to deform differ;
wherein the lower tray assembly includes a lower moldable component and a lower bracket, wherein required temperatures at which the lower moldable component and the lower bracket are heated to deform differ;
wherein the upper bracket and the lower bracket each have a front side edge configured to be near a labial side of the mouth of the patient and a rear side edge configured to be near a lingual side of the mouth of the patient, at least the upper bracket having a protruding front wall on the front side edge and a protruding rear wall on the rear side edge, and at least the lower bracket having a protruding rear wall on the rear side edge,
wherein the protruding front wall and the protruding rear wall of the upper bracket each comprises a clamping section formed by the respective protruding front wall and the protruding rear wall, the clamping section being configured to be positioned adjacent to a molar region when placed inside the mouth of the patient;
wherein at least a portion of the protruding front wall of at least the upper bracket is inclined towards the lingual side;
wherein, at a median sagittal plane, an inner wall of the protruding front wall of at least the upper bracket forms a lingually-inclined angle of less than 90° with a horizontal plane; and
wherein a length of the clamping section is greater than or equal to 20 mm.

30. The mandibular advancement device according to claim 29, wherein the upper bracket and the lower bracket are respectively configured to engage and support the upper moldable component and the lower moldable component.

31. The mandibular advancement device according to claim 29, wherein a connection module is provided between the upper bracket and the lower bracket.

32. The mandibular advancement device according to claim 31, wherein the upper bracket and the lower bracket each have a bottom wall configured to be opposite an occlusal surface of teeth when the patient bites, and the connection module includes a position adjustment snap-fit provided on the bottom wall.

33. The mandibular advancement device according to claim 31, wherein the connection module includes a protrusion provided on the protruding front wall and/or protruding rear wall of the upper bracket and/or the lower bracket.

34. A mandibular advancement device with enhanced stability, comprising:
an upper tray assembly, configured to be positioned adjacent to an upper dental arch of a patient when placed inside a mouth of the patient;
a lower tray assembly, configured to be positioned adjacent to a lower dental arch of the patient when placed inside the mouth of the patient;
wherein the upper tray assembly includes an upper moldable component and an upper bracket, wherein required temperatures at which the upper moldable component and the upper bracket are heated to deform differ;
wherein the lower tray assembly includes a lower moldable component and a lower bracket, wherein required temperatures at which the lower moldable component and the lower bracket are heated to deform differ;
wherein the upper bracket and the lower bracket each have a front side edge configured to be near a labial side of the mouth of the patient and a rear side edge configured to be near a lingual side of the mouth of the patient, at least the upper bracket having a protruding front wall on the front side edge and a protruding rear wall on the rear side edge, and at least the lower bracket having a protruding rear wall on the rear side edge;
wherein the protruding rear wall of at least the lower bracket includes a baffle section at a median sagittal plane;
wherein an angle between the inner wall of the protruding front wall of at least the upper bracket at a median sagittal plane and a horizontal plane is less than 90°; and
wherein a length of the baffle section is greater than or equal to 5 mm, and a height of the baffle section is less than or equal to 15 mm;
wherein each of the upper bracket and the lower bracket further comprise a clamping section formed by the protruding front wall and the protruding rear wall that is configured to be positioned near a molar area when placed inside the mouth of the patient; and
wherein the front wall and/or the rear wall of the upper bracket has a downward protrusion and the front wall and/or the rear wall of the lower bracket has an upward protrusion configured to maintain a positional relationship between the upper tray assembly and the lower tray assembly.

35. The mandibular advancement device according to claim 34, wherein the upper tray assembly and the lower tray assembly are in a form that is higher in a middle and lower on both sides.

36. The mandibular advancement device according to claim 34, wherein in the lower tray assembly, a maximum height of the baffle section is less than or equal to a maximum height of the lower moldable component.

37. The mandibular advancement device according to claim 34, wherein the upper tray assembly and the lower tray assembly are made of at least two different materials.

38. The mandibular advancement device according to claim 34, wherein a projected area of the upper moldable component on a horizontal plane is greater than a projected area of the upper bracket on the same plane, and a projected area of the lower moldable component on a horizontal plane is greater than a projected area of the lower bracket on the same plane.

39. The mandibular advancement device according to claim 34, wherein a height of the front wall of at least the upper bracket is less than 19.5 mm
